(19) United States
(12) Patent Application Publication
(10) Pub. No.: US 2012/0041544 A1
(43) Pub. Date: Feb. 16, 2012

(54) STENT GRAFT FENESTRATION

(75) Inventor: Yehuda G. Wolf, Mevasseret Zion (IL)

(73) Assignee: The Medical Research, Infrastructure and Health Services Fund of the Tel Aviv Medical Center, Tel-Aviv (IL)

(21) Appl. No.: 13/262,657

(22) PCT Filed: Apr. 1, 2010
(86) PCT No.: PCT/IB10/51438

§ 371 Date: Oct. 3, 2011

Related U.S. Application Data
(60) Provisional application No. 61/165,946, filed on Apr. 2, 2009.

(51) Int. Cl. A61F 2/82

(52) U.S. Cl. 623/1.35

(57) ABSTRACT

A method of deploying a stent graft in a blood vessel with at least one side branch vessel, comprising: (a) forming a blood flow conduit between the branch vessel and the blood vessel such that a vessel section of the conduit is axially located inside the blood vessel and a branch section is positioned inside the branch vessel; (b) deploying the stent graft inside the blood vessel so that blood flow into said at least one side branch is blocked by the stent graft and continues through the blood flow conduit; (c) forming an opening suitable for allowing blood flow between the blood vessel and the branch vessel; and (d) interrupting blood flow in the vessel section of the conduit.

### RELATED APPLICATION/S

This application claims the benefit of U.S. Provisional Application No. 61/165,946 filed on 2 Apr. 2009 the disclosure of which is incorporated herein by reference in its entirety. The contents of all of the above documents are incorporated by reference as if fully set forth herein.

### FIELD AND BACKGROUND OF THE INVENTION

The present invention, in some embodiments thereof, relates to endovascular procedures and, more particularly, but not exclusively, to a device and a method for deployment of a stent graft in a blood vessel including at least one branch vessel.

In-situ fenestration may be used during endovascular repair of a blood vessel at junctions with branch vessels. A typical procedure may involve placing a stent graft inside the vessel during which time one or more junctions are covered by the stent. During this time, blood flow into a branch vessel is interrupted, potentially exposing a patient undergoing the endovascular procedure to risk of complications and even death. Once the stent graft is in place, fenestration may be performed at each of the one or more junctions allowing blood flow to be restored into the one or more branch vessels.

Methods are known in the art for maintaining blood flow into the branch vessel during the endovascular procedure. Some methods may include use of a prefenestrated stent graft. Others may include separate conduits for allowing blood flow into the branch vessel. Two examples of the latter are described below.

WO 2007/082343 A1 “METHOD AND DEVICE FOR GRAFT FENESTRATION” relates to, “The present invention provides a method of creating fenestrations in situ through a body wall of a covered stent or endograft lumen. The fenestration is aligned with a side branch of the body lumen. The created fenestration of the graft (4) is in communication with a side branch. That is the patent or open side branch permits fluid communication from the main lumen across the stented or endograft lumen. Tools (41, 61, 51, 51B, 62) are described to carry out these fenestrations for either a graft (4) or a side branch that is communication with a graft (4) in vivo. Further these tools are described to carry out these methods for both in situ branch tissue and graft fenestration and alignment of the fenestrations The present invention provides a method for in situ fenestration, the method including the steps of: positioning a graft (4) or graft unit (4) in situ within a body lumen (1) and forming an initial void or space (6) between graft unit (4) and the inner wall (11) of the body lumen (1). The present invention also provides a catheter (41, 61) including a: a) a distal tip (62, 51, 51B) that can perform a piercing and opening action; and or b) a distal tip (62, 51, 51B) rotates by means of a drive shaft (52, 53) within the catheter (61) to cut and or open a tissue or graft (4) to form fenestration; and or c) a stabilization means (55, 42A, 42B, 51, 51B) to stabilize the position of the catheter (41, 61) during fenestration.”

Ohrlander et al. disclose in “The Chimney Graft: A Technique for Preserving or Rescuing Aortic Branch Vessels in Stent-Graft Sealing Zones”, Journal of Cardiovascular Therapy; August 2008; 15, 427-432; “A covered stent is deployed parallel to the main aortic stent-graft, protruding somewhat proximally, like a chimney, to preserve flow to a vital side branch covered by the aortic stent-graft. Use of a chimney graft makes it possible to use standard off-the-shelf stent-grafts to instantly treat lesions with inadequate fixation zones, providing an alternative to fenestrated stent-grafts in urgent cases, in aneurysms with challenging neck morphology, and for reconstituting an aortic side branch unintentionally compromised during endovascular repair. This technique has been used successfully in 10 patients, combining chimney grafts in the renal, superior mesenteric, left subclavian, left common carotid, and innominate arteries with stent-grafts in the abdominal (n=6) or thoracic (n=4) aorta. There has been no late chimney graft-related endoleak on imaging studies up to 8 months.”

### SUMMARY OF THE INVENTION

According to an aspect of some embodiments of the present invention there is provided a method of deploying a stent graft in a blood vessel with at least one side branch vessel, comprising (a) forming a blood flow conduit between the branch vessel and the blood vessel such that a vessel section of the conduit is axially located inside the blood vessel and a branch section is positioned inside the branch vessel, (b) deploying the stent graft inside the blood vessel so that blood flow into the at least one side branch is blocked by the stent graft and continues through the blood flow conduit, (c) forming an opening suitable for allowing blood flow between the blood vessel and the branch vessel, and (d) interrupting blood flow in the vessel section of the conduit.

According to some embodiments of the present invention, the method comprises forming the blood flow conduit with a stent adapted to transport blood from the blood vessel to the at least one side branch vessel. Optionally, the stent is a self-expanding stent. Optionally, the stent is a balloon-expandable stent.

According to some embodiments of the present invention, the method comprises parallely locating the vessel section between an inner wall of the blood vessel and the deployed stent graft.

According to some embodiments of the present invention, the method comprises forming the blood flow conduit such that the vessel section resists a compressive force exerted by the deployed stent graft.

According to some embodiments of the present invention, the method comprises interrupting blood flow in the vessel section by collapsing the vessel section. Optionally, the method comprises collapsing the vessel section under a compressive force exerted by the deployed stent graft when the stent graft is expanded. Optionally, the method comprises expanding the stent graft with an expandable balloon.

According to some embodiments of the present invention, the method comprises interrupting blood flow in the vessel section by mechanically manipulating the stent. Optionally, mechanically manipulating the stent comprises sliding a slip knot to cause disengagement of wires connected to the knot causing collapse of at least the vessel section. Optionally, mechanically manipulating the stent comprises pushing a joint to cause disengagement of wires connected to the joint causing collapse of at least the vessel section. Optionally, mechanically manipulating the stent comprises retrieving a rod supporting wires causing collapse of at least the vessel section.

According to some embodiments of the present invention, the method comprises forming the blood flow conduit such that blood flow through the conduit is uninterrupted when the branch section and the vessel section are angled with respect to one another. Optionally, an angle between the branch section and the vessel section ranges from 20°-180°.

According to some embodiments of the present invention, the method comprises forming the opening by initially inserting a fenestration device through the vessel section and making a perforation in the conduit and the stent graft. Optionally, the method comprises forming the opening by initially inserting a fenestration device through the stent graft and making a perforation in the conduit and the stent graft. Optionally, the method further comprises inserting an expansion balloon into the perforation and expanding the balloon to increase the size of the perforation to that of the opening. Optionally, the method comprises inserting the balloon from the branch vessel. Optionally, the method comprises inserting the balloon from the blood vessel.

According to some embodiments of the present invention, the method comprises leaving at least a portion of the conductor inside the blood vessel and/or branch vessel. Optionally, the method comprises biodegrading the at least a portion of the conductor. According to an aspect of some embodiments of the present invention there is provided a blood flow conductor comprising a vessel section and a branch section connected together by a bendable section, wherein at least the vessel section is adapted to be collapsed after deployment in the aorta and is strong enough to maintain structural rigidity when compressed between a deployed stent graft and the aortic wall.

According to some embodiments of the present invention, the blood flow conductor comprises a stent. Optionally, the stent is a self-expanding stent. Optionally, the stent comprises one or more wires configured to act as struts for maintaining the structural rigidity. Optionally, the one or more wires are coil shaped. Optionally, the one or more wires comprise a shape memory alloy.

According to some embodiments of the present invention, the stent is a balloon-expandable stent. Optionally, the vessel section of the stent is pliable.

According to some embodiments of the present invention, the blood flow conductor comprises a sheath for transporting the stent to a location for deployment in the aorta. Optionally, the stent is adapted to be folded into the sheath following collapse of at least the vessel section.

According to some embodiments of the present invention, the vessel section is adapted to be axially displaced in the aorta. Optionally, the branch section is adapted to be inserted in a branch vessel. Optionally, the bendable section is adapted to bend through an angle ranging from 20°-180°. Additionally or alternatively, the bendable section is kink-resistant.

According to some embodiments of the present invention, the blood flow conductor comprises a wire adapted to collapse the vessel section when pulled in a proximal direction. Optionally, the blood flow conductor comprises a sliding knot for collapsing the vessel section. Optionally, the blood flow conductor comprises a breakable joint for collapsing the vessel section. Additionally or alternatively, the blood flow conductor comprises a hook for collapsing the vessel section.

According to some embodiments of the present invention, the blood flow conductor comprises at any one point a circular cross-section.

According to some embodiments of the present invention, the blood flow conductor comprises a diameter ranging from 3 mm-15 mm.

According to an aspect of some embodiments of the present invention there is provided a medical kit comprising an aortic stent graft; and a blood flow conductor comprising a vessel section and a branch section connected together by a bendable section, wherein at least the vessel section is adapted to be collapsed after deployment in the aorta and is strong enough to maintain structural rigidity when compressed between a deployed stent graft and the aortic wall.

According to some embodiments of the present invention, the medical kit comprises a fenestration device for making a fenestration in the stent graft and the conductor. Optionally, the fenestration device comprises a catheter adapted to accommodate a needle for perforating the stent graft and the conductor. Optionally, the fenestration device includes at least one expandable balloon for bending a tip of the catheter.

According to some embodiments of the present invention, the medical kit comprises a guide wire for directing the fenestration device to a fenestration location. Optionally, the guide wire is adapted to perforate the stent graft and the conductor. Optionally, the guide wire is adapted to be inserted from the branch section of the conductor for perforating the stent graft and the conductor. Optionally, the guide wire is adapted to be inserted from the vessel section of the conductor for perforating the stent graft and the conductor. Additionally or alternatively, the guide wire is adapted to be inserted from the stent graft for perforating the stent graft and the conductor.

According to some embodiments of the present invention, the medical kit comprises a branch stent and/or a branch stent graft for inserting through the fenestration and in the branch vessel.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1-17 schematically illustrate an exemplary implementation of the method of deployment of an exemplary stent graft in a blood vessel including at least one branch vessel using an exemplary branching scaffold, according to an embodiment of the present invention;

FIGS. 18-33 schematically illustrate an exemplary implementation of the method of deployment of an exemplary stent graft in a blood vessel including at least one branch vessel, using an exemplary stent, according to some embodiments of the present invention;

FIGS. 34-36 schematically illustrate an exemplary branching scaffold and a method of loss of structural rigidity in the scaffold, according to some embodiments of the present invention;

FIGS. 37-40 schematically illustrate an exemplary branching scaffold and a method of loss of structural rigidity in the scaffold, according to some embodiments of the present invention;

FIGS. 41-43 schematically illustrate an exemplary branching scaffold and a method of loss of structural rigidity in the scaffold, according to some embodiments of the present invention;

FIG. 44 schematically illustrates an exemplary branching scaffold, according to some embodiments of the present invention;

FIG. 45 schematically illustrates an exemplary branching scaffold, according to some embodiments of the present invention;

FIG. 46 schematically illustrates a fenestration device, according to some embodiments of the present invention;

FIG. 47 schematically illustrates a fenestration device, according to some embodiments of the present invention;

FIG. 48 schematically illustrates a fenestration device, according to some embodiments of the present invention.

### DESCRIPTION OF EMBODIMENTS OF THE INVENTION

The present invention, in some embodiments thereof, relates to endovascular procedures and, more particularly, but not exclusively, to a device and a method for deployment of a stent graft in a blood vessel including at least one branch vessel.

An aspect of some embodiments of the present invention relates to a method of deploying a stent graft in a blood vessel including at least one branch vessel by, in the following order, (a) forming a conduit for blood flow from the blood vessel into the branch vessel, (b) deploying the stent graft inside the blood vessel, (c) forming an opening connecting the stent graft with the conduit to allow blood flow between the blood vessel and the branch vessel through the opening (fenestration), and (d) interrupting blood flow in a vessel section of the conduit. Optionally, the blood flow is interrupted by collapsing the vessel section internally. Optionally, the vessel section collapses to a thickness of less than 2 mm, less than 1.5 mm, less than 1 mm, less than 0.5 mm, less than 0.2 mm, less than 0.1 mm, less than 0.05 mm, less than 0.02 mm, less than 0.01 mm. Optionally, the stent graft is an aortic stent graft. Optionally, the stent graft is any type of stent graft used for endovascular repair of an aneurysm. Optionally, the stent graft is any type of stent graft used in endovascular treatment of stenosis. Optionally, the stent graft is any type of conductor having characteristics suitable for transporting blood from the blood vessel into the branch vessel, for example, from the aorta into any aortic branch vessel. Optionally, the conductor is a removable stent or a removable stent graft. Additionally or alternatively, the blood flow conduit is formed from the blood vessel into the branch vessel. Optionally, the blood flow conduit is formed from the branch vessel into the blood vessel. Optionally, a blood flow conduit is formed for each branch vessel in a blood vessel comprising a plurality of branch vessels. Optionally, at least a portion of the conductor is adapted to remain in the blood vessel (following vessel section collapse) and/or the branch vessel. Additionally or alternatively, the stent graft is adapted to biodegrade (following vessel section collapse).

In some embodiments, the blood flow conduit is formed by a blood flow conductor adapted to transport blood from the blood vessel to the branch vessel. Optionally, the blood flow conductor includes a stent. Optionally, the conductor includes a vessel section axially positioned inside the blood vessel and a branch section positioned inside the branch vessel. Additionally or alternatively, the vessel section and the branch section are joined together by a bendable section. Optionally, the conductor includes a length ranging from 3 cm-20 cm, for example, 3 cm-6 cm, 6 cm-10 cm, 10 cm-15 cm, 15 cm-20 cm. Optionally, a length of the vessel section and a length of the branch section are different. Optionally, the length of the vessel section and the branch section are the same. Additionally or alternatively, the length of the vessel section is less than 15 cm, less than 12 cm, less than 9 cm, less than 6 cm, less than 3 cm. Optionally, the length of the branch section is less than 15 cm, less than 12 cm, less than 9 cm, less than 6 cm, less than 3 cm. Optionally, a length of the bendable section is less than 10 cm, less than 6 cm, less than 3 cm, less than 1 cm.

In some embodiments, the blood flow conductor includes a metal coverage area ranging from 1%-35% of total surface coverage, for example 1%-10%, 10%-15%, 15%-25%, 25%-35%. Optionally, a total surface coverage ranges from 10%-95%, for example, 10%-25%, 25%-45%, 45%-70%, 70%-95%. Optionally, a strut thickness in the conductor ranges from 200 μm-800 μm, for example, 200 μm-350 μm, 350 μm-550 μm, 550 μm-800 μm. Optionally, a strut width in the conductor ranges from 200 μm-800 μm, for example, 200 μm-350 μm, 350 μm-550 μm, 550-800 μm. Optionally, strut thickness and/or strut width is greater than 800 200 μm-800 μm, for example, 200 μm-350 μm, 350 μm-550 μm, 550 μm-800 μm.

In some embodiments, the blood flow conductor includes solid walls so that there is no blood flow through the walls. Optionally, the conductor includes 100% surface coverage. Optionally, the conductor includes a biodegradable material. Optionally, the conductor includes a biocompatible material.

In some embodiments, the vessel section is parallely positioned inside the blood vessel between the deployed stent graft and an inner wall of the blood vessel. Optionally, the vessel section has a structural rigidity adapted to resist a compressive force radially exerted by the stent graft and by an inner wall of the blood vessel. Additionally or alternatively, the structural rigidity prevents deformation of the vessel section. Optionally, the vessel section maintains a patency. Optionally, the vessel section is adapted to lose structural rigidity and collapse under the compressive force exerted by the stent graft when expanded by an inflatable balloon placed inside the stent graft, hereinafter referred to as balloon-expanded stent. Optionally, collapse of the vessel section interrupts blood flow through the vessel section. Additionally or alternatively, the vessel section is adapted to collapse under the compressive force exerted by the stent graft when expanded by stent expanding means other than an inflatable balloon, for example, by the introduction of large diameter object (larger diameter than the stent graft) into the stent graft. Optionally, the vessel section is adapted to lose structural rigidity and/or collapse through mechanical manipulation, for example, by acting on mechanical elements forming the conductor such as, for example, a wire, a rod, a slip knot, a joint, a hook, or any combination thereof. Optionally, pulling or cutting at least one wire causes a loss of structural rigidity in the vessel section. Optionally, pulling or cutting the at least one wire causes the vessel section to collapse. Optionally, release of a slip knot causes loss of structural rigidity and/or collapse of the vessel section. Optionally, pulling or pushing on a rod causes loss of structural rigidity and/or collapse of the vessel section. Optionally, removal of a stiffener tube inserted in the vessel section causes loss of structural and/or collapse of the vessel section. Additionally or alternatively, exposure to body temperature and/or body fluids over a predetermined period of time causes the vessel section to lose structural rigidity and/or collapse. Optionally, structural rigidity is also lost in the branch section.

In some embodiments, the branch section is fitted inside the branch vessel and is adapted to adhere to the inner wall of the branch vessel. Optionally, the branch section is adapted to remain stationary inside the branch vessel. Optionally, the branch section includes a lumen for accommodating a branch vessel stent graft. Optionally, a branch stent graft is implanted in the branch vessel following fenestration of the branch section and the stent graft.

In some embodiments, the bendable section is adapted to bend through an angle greater than 90°, for example, in a range between 0°-100° inclusive, 0°-120° inclusive, 0°-150° inclusive, 0°-180 inclusive, without kink so that blood flow through the section is uninterrupted regardless of the angle of bend. °. Optionally, the bendable section is adapted to bend through an angle in a range from 20°-180°, for example 20°-160°. Additionally or alternatively, bending of the bendable section without kink includes a reduction in the cross-sectional area of the section of less than 10%, less than 20%, less than 30%, less than 40%, less than 50%, less than 60%. Optionally, the bendable section is suitable for fenestration.

In some embodiments, the blood flow conductor includes a circular cross section at any point along its whole length. Optionally, the conductor is cylindrically shaped. Optionally, a diameter of the conductor ranges from 3 mm-15 mm, for example 3 mm-5 mm, 5 mm-8 mm, 8 mm-12 mm, 12 mm-15 mm. Optionally, the vessel section diameter is of a first diameter and the branch section of a different second diameter. Optionally, the cross-sectional shape of the conductor is non-circular, for example, elliptical, oval.

In some embodiments, the blood flow conduit is formed by a self expanding stent. This self expanding stent, which may be also be referred to as a “branching scaffold”, may be made from a metal, for example, an elastic metal such as a shape memory alloy. Optionally, the shape memory alloy includes nitinol and/or stainless steel. Optionally, the branching scaffold includes any bio-compatible and/or bio-degradable material. The branching scaffold may include one or more wires adapted to act as struts for enabling the vessel section to resist the compressive force of the stent graft. Optionally, the one or more wires are coil-shaped (helical). Optionally, the one or more wires are intertwined. Optionally, the one or more wires are spaced for accommodating the fenestration. Optionally, the one or more wires are arranged in any shape or form suitable for resisting the compressive force of the stent graft and the inner wall, enable fenestration, and allow for collapsing of the vessel section as previously described. Additionally or alternatively, the branching scaffold includes the bendable section joining the vessel section of the scaffold with the branch section of the scaffold. Optionally, a branch stent graft inserted through the fenestration is accommodated inside the branch section.

In some embodiments, the branching scaffold includes a single coiled wire. Optionally, the branching scaffold includes 2-4 coiled wires. Optionally, the number of coiled wires is greater than 4, for example 6-10 wires, 10-20 wires, 20-50 wires, 50-100 wires, or more. Optionally, the scaffold includes a central rod which connects the wires together by a single loop (slip knot) at a distal end of the rod, allowing the wires to disconnect from one another when the knot slides. Optionally, the central rod connects the wires together by a breakable joint allowing the wires to be disconnected from one another. Optionally, the wires are connected together to the rod by a removable hook at the distal end which allows for the wires to disconnect from one another. Additionally or alternatively, the central rod is a wire. Optionally, the wires are connected to the rod by a plurality of loops at different locations along the rod.

In some embodiments, the branching scaffold is included in a sheath or sleeve adapted to be inserted into the blood vessel. Optionally, the sheath includes stiffness substantially greater than that of the branching scaffold. Optionally, the sheath is of an external diameter ranging from 1 mm-6 mm, for example 1 mm-2 mm, 2 mm-3 mm, 3 mm-4 mm, 4 mm-5 mm, 5 mm-6 mm. Optionally, the branching scaffold is deployed by extending the rod out of the sheath in a distal direction through the blood vessel and through the junction into the branch vessel. Optionally, the deployment may be through the branch vessel and through the junction into the blood vessel. Additionally or alternatively, the branching scaffold may be retrieved by pulling the rod in a proximal direction into the sheath. Optionally, the branching scaffold is adapted to collapse onto itself so that its diameter is reduced to a dimension suitable to fit the scaffold into the sheath when retrieved. Additionally or alternatively, the branching scaffold is adapted to deform so that the wires and the rod are disconnected from one another, allowing the wires to substantially straighten for retrieving into the sheath. Optionally, only a portion of the wires is retrieved. Optionally, the portion of the wires is those in the branch section. Optionally, the wires remain connected to the rod at a proximal end of the rod.

In some embodiments, retrieving the rod causes the vessel section to loose structural rigidity. Optionally, the branch section loses structural rigidity. Optionally, structural rigidity is lost by sliding at least one slip knot (optionally pulling the rod in a proximal direction and the knot slips out). Optionally, structural rigidity is lost by cutting the rod. Optionally, structural rigidity is lost by pushing the rod in a distal direction and breaking a union of the wires to a joint at a distal end of the rod. Optionally, twisting of the rod will cause the wires to unhook from a hook on a distal end of the rod. Additionally or alternatively, structural rigidity is lost by cutting at least one of the wires. Optionally, structural rigidity is lost by pulling at least on one of the wires. Optionally, for branching scaffolds including a thermal shape memory alloy, a temperature change may be induced in the scaffold for causing loss of structural rigidity; for example, a balloon filled with a saline at a suitable temperature may be inserted into the vessel section to cause a reduction in resilience. Optionally, for a biodegradable branching scaffold or a soluble scaffold, structural rigidity is lost after a predetermined period of time has passed.

In some embodiments, the blood flow conduit is formed by a balloon-expandable stent. Optionally, a bendable balloon is used to expand the stent. Optionally, the bendable balloon is bendable through the bending angle of the bendable section of the stent. Optionally, the balloon-expandable stent is expanded by stent-expanding means other than a balloon. Optionally, the stent includes materials similar to that in the in the branching scaffold. Optionally, the stent includes a structural rigidity similar to that of the branching scaffold. Additionally or alternatively, the stent includes a vessel section which is substantially pliable, allowing the section to substantially flatten under the compressive force of the balloon-expanded stent graft in the blood vessel so as to interrupt blood flow through the vessel section. Optionally, the vessel section loses structural rigidity by cutting and/or pulling wires included in the stent. Optionally, the vessel section loses structural rigidity by sliding a slip knot. Additionally or alternatively, structural rigidity is lost by removing a rod included in the stent. Optionally, retrieval of at least a portion of the stent causes loss of structural rigidity in the vessel section. Optionally, the vessel section is occluded by a balloon or other implantable occlusion means. Optionally, the stent is included in a sheath which is inserted into the blood vessel. Optionally, the sheath is inserted from the branch vessel.

In some embodiments, the blood flow conduit is formed by a stent graft including a fabric or other impervious material such as, for example, Dacron, PTFE, and the like. Optionally the stent characteristics of the stent graft are similar to that of the branching scaffold or the balloon-expandable stent. Optionally, wires in the stent graft have a cross-section so that the aortic wall in not damaged by the wires pressing against it. Additionally or alternatively, the stent graft includes a plain fabric tube. Optionally, the branch section of the stent graft and the stent graft in the blood vessel are anatomosed. Optionally, the vessel section of the stent graft is substantially flattened by the compressive force exerted by the balloon-expanded stent graft in the blood vessel interrupting blood flow through the vessel section. Optionally, blood flow in the vessel section is interrupted by inserting a one way valve into the vessel section following fenestration. Optionally, the valve is adapted to disconnect the fenestration and the branch section of the stent graft from the vessel section to prevent blood flow into the blood vessel through the vessel section. Optionally, blood flow in the vessel section is interrupted with a balloon or other implantable occlusion device. Additionally or alternatively, the stent is a self-expanding stent. Optionally, the stent is a balloon-expandable stent.

In some embodiments, the blood flow conduit is formed by a biodegradable, soluble, or bio-modifiable stent (e.g. Poly-l-Lactic Acid or other polymer or erodible, biodegradable metal such as magnesium). Optionally, the stent is a self-expanding stent or a balloon-expandable stent with structural rigidity similar to that of the branching scaffold or the balloon-expandable stent. Optionally, the stent is designed so that its structural rigidity is maintained for a relatively short period of time such as, for example several minutes (for example 15 minutes), an hour, a number of days, or more. Optionally, rapid modification of the structural rigidity of the stent is desired for reducing the stiffness or resilience of vessel section, thereby enabling it to collapse under the force of expanded stent graft in the blood vessel following fenestration. Additionally or alternatively, a stent material is stretchable, tearable or breakable to accommodate the opening. Optionally, substantially complete biodegradation, dissolution and absorption extends over a longer period of time, such as days or months.

In some embodiments, fenestration is performed under fluoroscopy and angiography, using techniques know in the art. Optionally, the fenestration may be opened in a direction from the stent graft in the blood vessel towards the blood flow conduit. Optionally, the fenestration is opened in a direction from the blood flow conduit towards the stent graft in the blood vessel. Additionally or alternatively, the fenestration is located in the bendable section of the blood flow conduit positioned at the junction of the branch vessel with the blood vessel. Optionally, the bendable section is punctured with a small hole which is then expanded to a size of the fenestration. Optionally, a fenestration device includes a catheter with a retractable (movable) sharp needle to perform the puncture at the site of the opening. Optionally, the needle is a bent hollow needle. Optionally, the needle is a Rosch-Uchida/Ushida needle. Optionally, a guide wire is passed through the needle. Optionally, a stiff guidewire may be used in lieu of the needle. Optionally, the catheter includes a straight shape when in a neutral position. Optionally, the fenestration device is inserted through the same sheath including the branching scaffold or the stent (optionally, a same sheath is used for the branching scaffold and the balloon-expandable stent). Optionally, the fenestration device includes a mechanism for bending the tip of the catheter and pushing it against the wall of the stent graft at the point intended for fenestration. Optionally, the mechanism includes a balloon which extends beyond the tip of the catheter and is adapted to bend the tip. Additionally or alternatively, several balloons are configured asymmetrically to push the catheter tip to one side. Optionally, a small balloon is located on a side of the catheter towards which the catheter bends, and one or more longer balloons are located on an opposing side of the catheter, extending beyond the catheter tip. Additionally or alternatively, the catheter includes one or more wires running along one aspect of its wall. Optionally, the catheter is straight when the wires are in a neutral position. Optionally, the catheter tip is bent to one side when the wires are pushed forward in a distal direction. Additionally or alternatively, the catheter tip is bent when the wires are pulled in a proximal direction. Optionally, the needle may be advanced through the catheter when the catheter tip is at the location of the fenestration for penetrating the graft material. Additionally or alternatively, a guidewire is passed through the needle and into the stent graft.

In some embodiments, the blood flow conductor is included in a medical kit for deploying a stent graft in a blood vessel with at least one branch vessel. Optionally, the medical kit includes at least the needle of the fenestration device. Optionally, the fenestration device is included. Additionally or alternatively, a stent graft is included in the medical kit. Optionally, the stent graft is an aortic stent graft. Optionally, the stent graft is any type of stent graft used for endovascular repair of an aneurysm. Optionally, the stent graft is any type of stent graft used in endovascular treatment of stenosis.

In some embodiments, a procedure for deploying the stent graft in a blood vessel including at least one branch vessel includes the following steps:

(a) Form the blood flow conduit by inserting a blood flow conductor through the junction so that the vessel section is positioned inside the blood vessel and the branch section is in the branch vessel. Insertion of the conduit may be from the blood vessel or from the branch vessel, as determined by the physician. Optionally, the conduit includes the branching scaffold. Optionally, the conduit includes the balloon-expandable stent. Optionally, the conduit includes the stent graft including a fabric or other impervious material such as, for example, Dacron, PTFE, and the like. Optionally, the conduit includes the biodegradable, soluble, or bio-modifiable stent. Additionally or alternatively, for more than one branch vessel, use one conduit of the same type for each branch vessel. Optionally, any combination of the different types of conduits may be used for the plurality of branch vessels.
(b) Deploy the stent graft in the blood vessel. Optionally, align the vessel section so that it is parallel to the stent graft. Optionally, align the vessel section so that the opening through which blood flows into the vessel section extends beyond the opening through which blood flows into the stent graft.
(c) Insert the fenestration device and manipulate it so that the tip is at the location where the opening is to be made. Optionally, the fenestration device is inserted through the sheath. Optionally, the fenestration device is inserted externally to the sheath. Optionally, the fenestration device is inserted into the conduit from the vessel section. Optionally, the fenestration device is inserted into the conduit from the branch section. Additionally or alternatively, the fenestration device is inserted through the stent graft in the blood vessel. Optionally, the location of the fenestration is in the bendable section.
(d) Perforate the location of the fenestration by pushing the needle in the fenestration device. Optionally, the stiff guide wire is used to perforate. Optionally, the perforation is made through both the blood flow conductor and the stent graft in the blood vessel. Optionally, the perforation is made only through the stent graft (as is the case with the branching scaffold). Optionally, methods other than that described herein in step (d), (e), and (f) may be used for fenestration.
(e) Manipulate the guide wire so that it passes through the perforation. Optionally, the guide wire is passed from the vessel section or the branch section into the lumen of the stent graft in the blood vessel. Optionally, the guide wire is passed from the lumen of the stent graft in the blood vessel into the vessel section or branch section.
(f) Enlarge the fenestration using an instrument adapted to expand the size of the opening to the required size (optionally a size of the branch vessel). Optionally, the mechanism includes an expandable balloon. Optionally, the instrument is guided to the opening using the guide wire.
(g) Interrupt blood flow through the vessel section. Optionally, blood flow is interrupted by causing a loss of structural rigidity in the vessel section and expanding the stent graft in the blood vessel. Optionally, the stent graft is expanded by an expandable balloon. Optionally, the loss of structural rigidity is caused by retrieval of the conductor. Optionally, the wire is cut in the conductor. Optionally, the rod is removed in the conduit. Additionally or alternatively, the slip knot is slid in the conduit. Optionally, joint connection to the wires is broken. Optionally, the wires are unhooked. Optionally, partial or whole bio-degradation, bio-dissolution, or bio-modification, or any combination thereof, occurs in the conduit. Optionally, interrupt blood flow in the vessel section using other non-collapsible means, such as, for example, inserting the one-way valve, inserting a balloon, or inserting other implantable means of occlusion. Optionally, blood flow in the vessel section may be interrupted by inserting a branch stent graft into the branch vessel.
(h) Optionally, insert a branch stent graft through the fenestration by guiding along a guide wire. Optionally, a stent is inserted. Optionally, the branch stent graft is inserted from the stent graft in the blood vessel. Optionally, the branch stent graft is inserted through the branch vessel in a direction towards the stent graft in the blood vessel. Optionally, the branch stent graft is expanded by unsheathing, balloon expansion, or other suitable method, to create a sealed branch with the stent graft. Optionally, the branch stent graft is manipulated from the direction of the stent graft in the blood vessel. Optionally, the branch stent graft is a balloon-expandable stent graft. Optionally, the balloon is inserted through the fenestration from the stent graft in the blood vessel.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

Before explaining at least one embodiment of the invention in detail, it is to be understood that the invention is not necessarily limited in its application to the details set forth in the following description. The invention is capable of other embodiments or of being practiced or carried out in various ways.

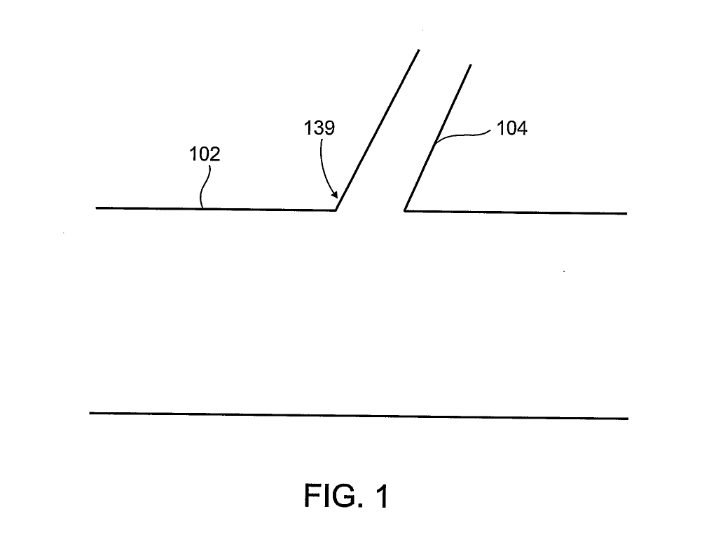

Referring now to the drawings, FIGS. 1-17 schematically illustrate an exemplary implementation of the method of deployment of an exemplary stent graft 116 in a blood vessel 102 including at least one branch vessel 104 using an exemplary branching scaffold 100, according to an embodiment of the present invention.

FIG. 1 shows blood vessel 102 with branch vessel 104 connected to the blood vessel at junction 139. In some embodiments, blood vessel 102 is the aorta. Optionally, blood vessel 102, branch vessel 104 and junction 139 include the aortic bifurcation into the iliac arteries; the ascending aorta and coronary arteries; the aortic arch and great vessels; the abdominal aorta and visceral and renal branches; or the iliac bifurcation into internal and external iliac or other arterial branch points.

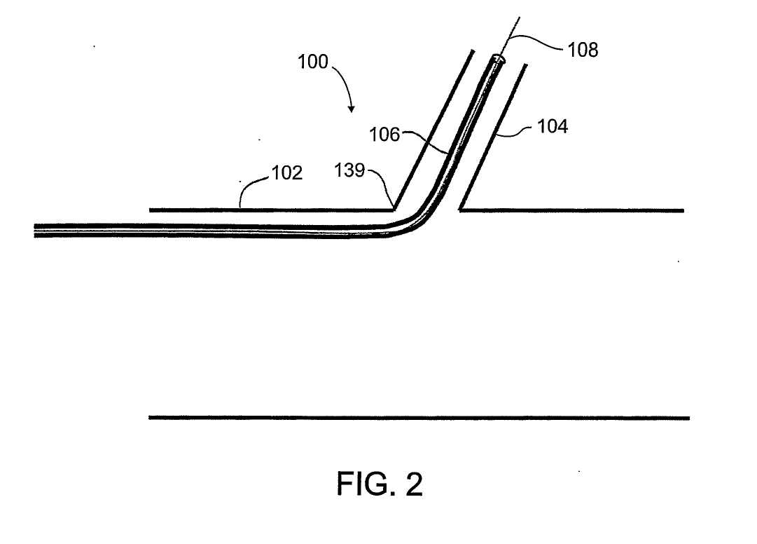

FIG. 2 shows an exemplary sheath 106 inserted into blood vessel 102 and through junction 139 into branch vessel 104. Optionally, sheath 106 includes a rod 108 adapted to deploy branching scaffold 100. Optionally, rod 108 includes a wire.

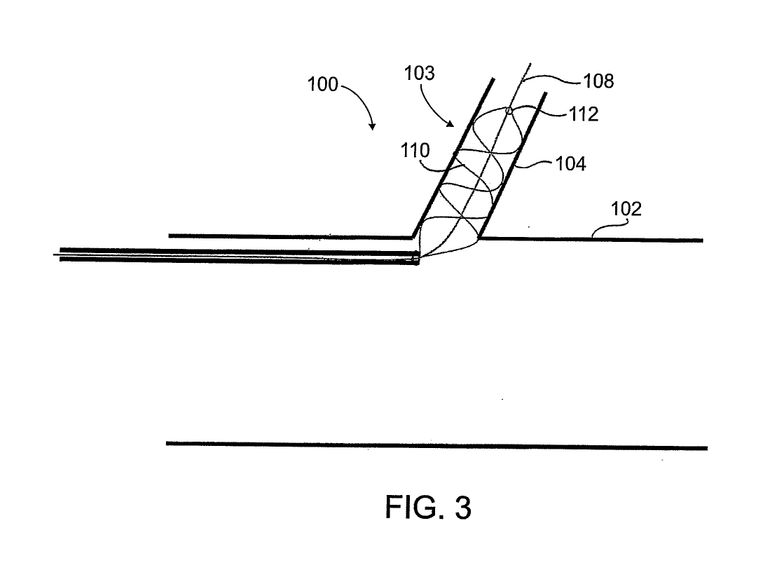

FIG. 3 shows a partial deployment of branching scaffold 100. Optionally, an exemplary branch section 103 of branching scaffold 100 is deployed inside branch vessel 104. Optionally, branching scaffold is formed from wires 110. Optionally, wires 110 are connected together to rod 108 at loop 112 on a distal end of the rod.

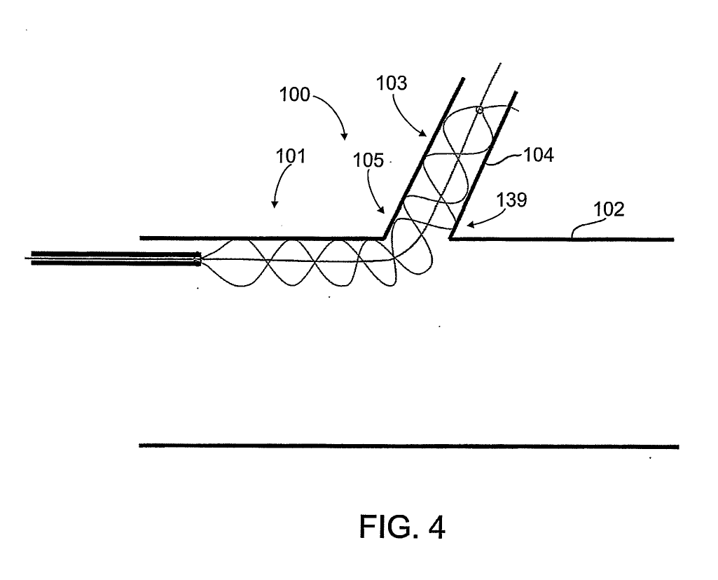

FIG. 4 shows a full deployment of branching scaffold 100. Optionally, branch section 103 is deployed in branch vessel 104, an exemplary vessel section 101 is axially deployed in blood vessel 102, and an exemplary bendable section 105 joining the vessel section and branch section is deployed at junction 139. Optionally, vessel section 101 is parallel to stent graft 116.

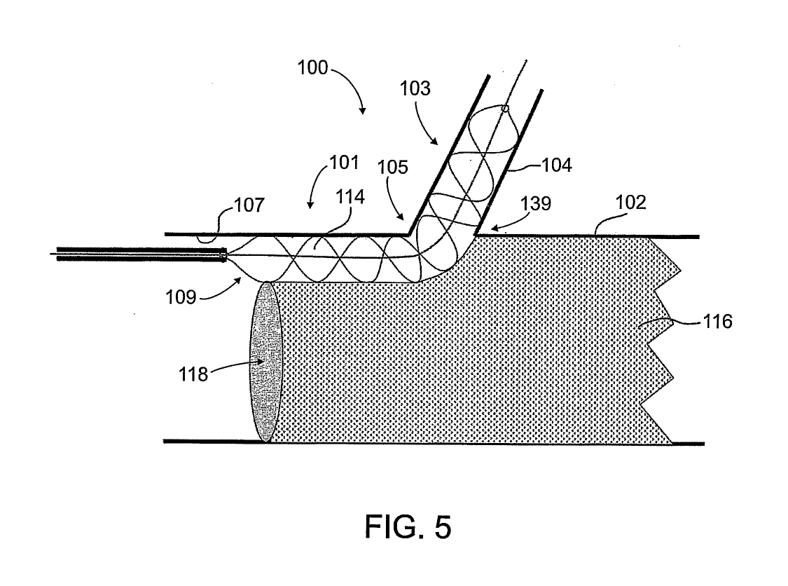

FIG. 5 shows a deployment of stent graft 116 in blood vessel 102, following deployment of branching scaffold 100. Optionally, stent graft 116 pushes vessel section 101 against an inner wall 107 of blood vessel 102. Optionally, wires 110 acts as struts and provide vessel section 101 with a structural rigidity for resisting a compressive force radially exerted on the section by stent graft 116 and inner wall 107. Optionally, a conduit lumen 114 is formed between stent graft 116 and inner wall 107 extending from a conduit opening 109 and extending into branch vessel 104 for conducting blood flow from blood vessel 102 through junction 139 into the branch vessel. Optionally, blood flows into a lumen 118 in stent graft 116.

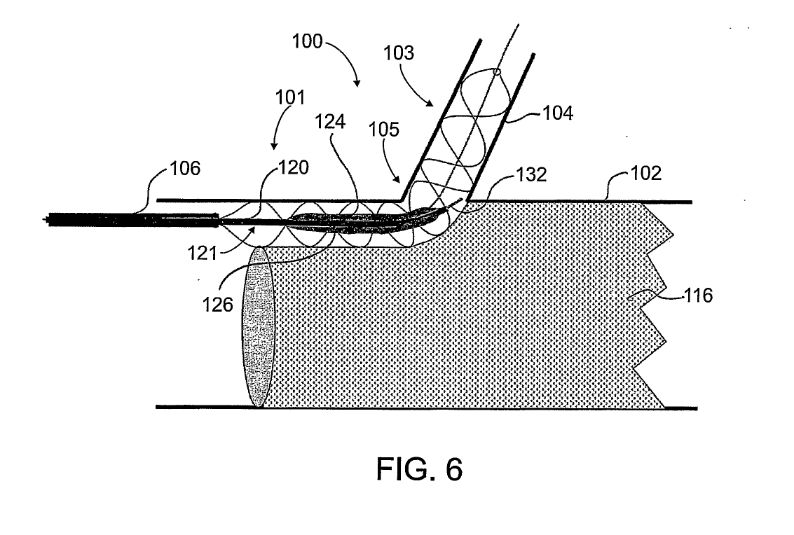

FIG. 6 shows an optional deployment of an exemplary fenestration device 121 for perforating a hole to form a fenestration in bendable section 105 and stent graft 116. Optionally, fenestration device 121 is inserted through sheath 106 and is manipulated through vessel section 101 to bendable section 105 by a guide wire 132. Optionally, fenestration device 121 can reach bendable section 105 from other directions, for example, from branch vessel 104. Optionally, a location where the opening is to be made in stent graft 116 (opposite bendable section 105) can be reached through blood vessel 102. Optionally, fenestration device 121 is guided to the location in bendable section 105 where the opening is to be made. Optionally, fenestration device 121 is manipulated into vessel section 101 without being inserted in sheath 106. Optionally, fenestration device 121 includes an exemplary catheter 120 and two expandable balloons at a distal end, a small balloon 126 on one side of the catheter and an opposing larger balloon 124.

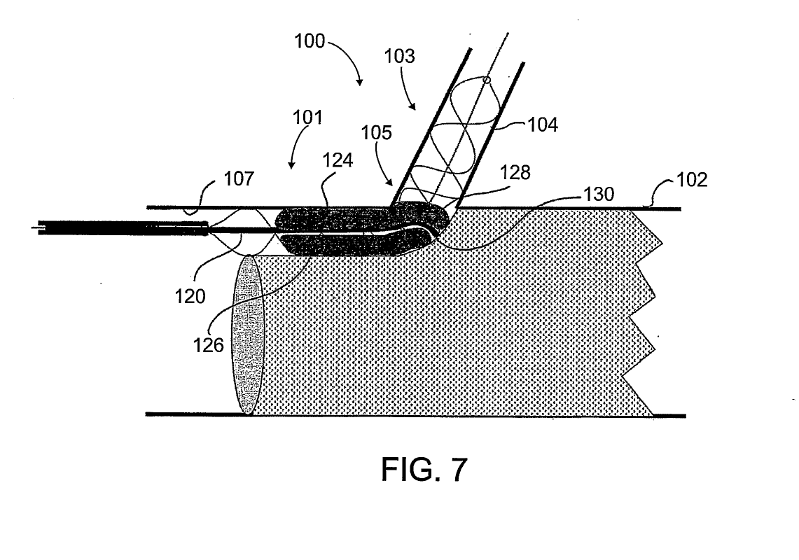

FIG. 7 shows a catheter tip 128 in catheter 120 positioned at a fenestration location 130 in bendable section 105. Optionally, catheter tip 128 is bent to position at fenestration location 130 by first expanding small balloon 126 and then large balloon 124. Optionally, large balloon 124 is supported on one side by inner wall 107 and by small balloon 124 on an opposing side for bending catheter tip 128.

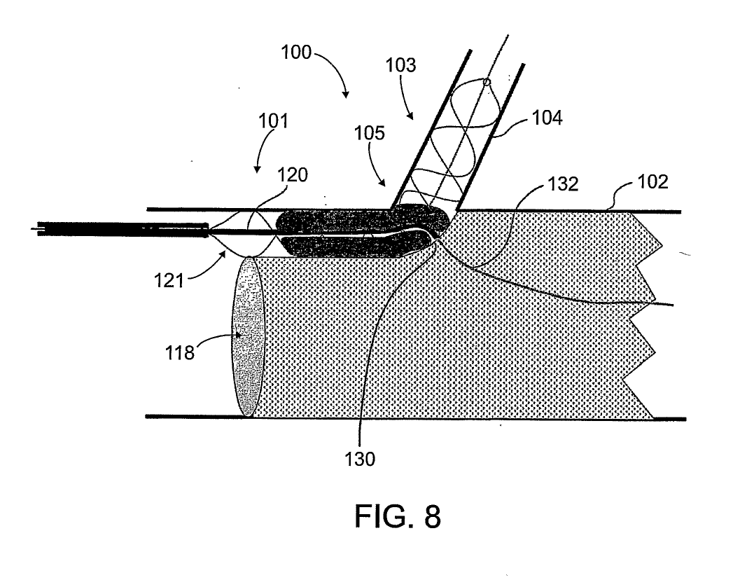

FIG. 8 shows fenestration device 121 having perforated stent graft 116 at fenestration location 130. Optionally, guide wire 132 is passed through the opening from catheter 120 inside vessel section 101 to lumen 118 in stent graft 116.

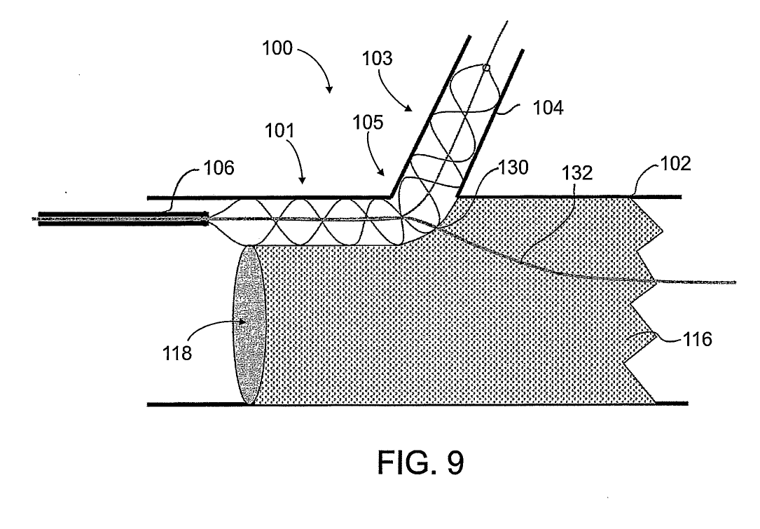

FIG. 9 shows catheter 120 retrieved from sheath 106. Optionally, guide wire 132 extends through vessel section 101 and through the opening in fenestration location 130 in bendable section 105, passing into lumen 118 in stent graft 116.

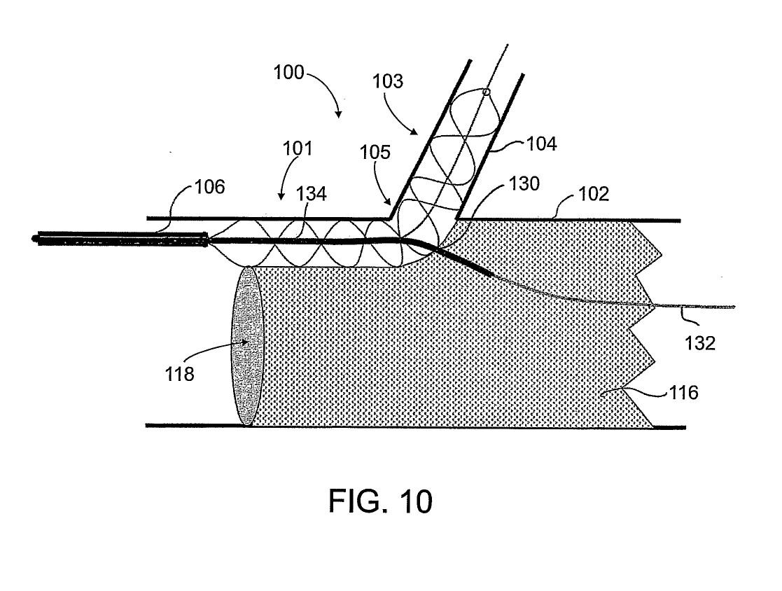

FIG. 10 shows an exemplary balloon catheter 134 inserted through sheath 106 and guided by guide wire 132 through the opening at fenestration location 130. Optionally, a distal end of balloon catheter 134 extends into lumen 118 in stent graft 116.

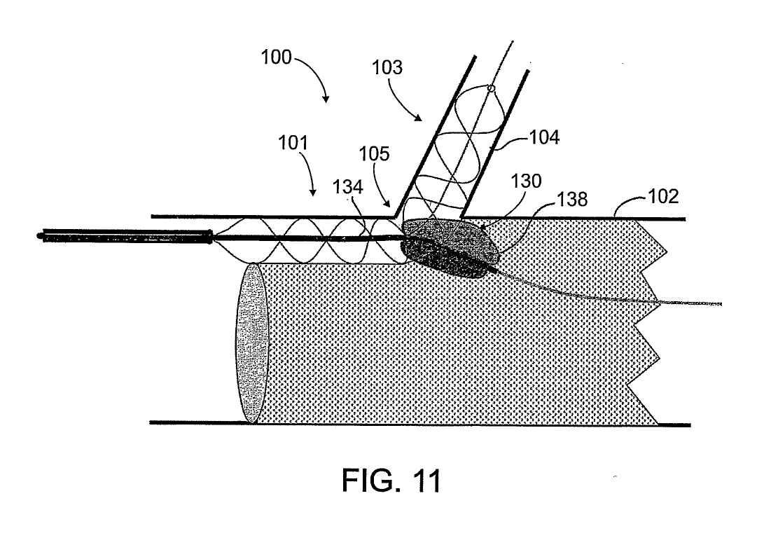

FIG. 11 shows an exemplary balloon 138 in balloon catheter 134 being expanded in the hole at fenestration location 130. Optionally, balloon 138 expands the hole in fenestration location 130 to a suitable size for allowing blood flow through the hole between branch vessel 104 and blood vessel 102.

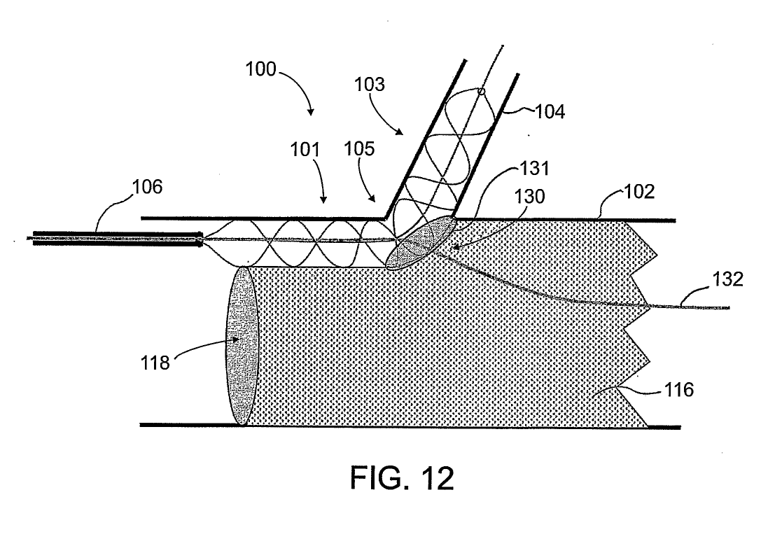

FIG. 12 shows an exemplary suitable opening 131 at fenestration location 130 for allowing blood flow between branch vessel 104 and blood vessel 102. Optionally, balloon catheter 134 retrieved from sheath 106. Optionally, guide wire 132 remains in place, passing through vessel section 101 and bendable section 105, through opening 131 and into lumen 118 in stent graft 116.

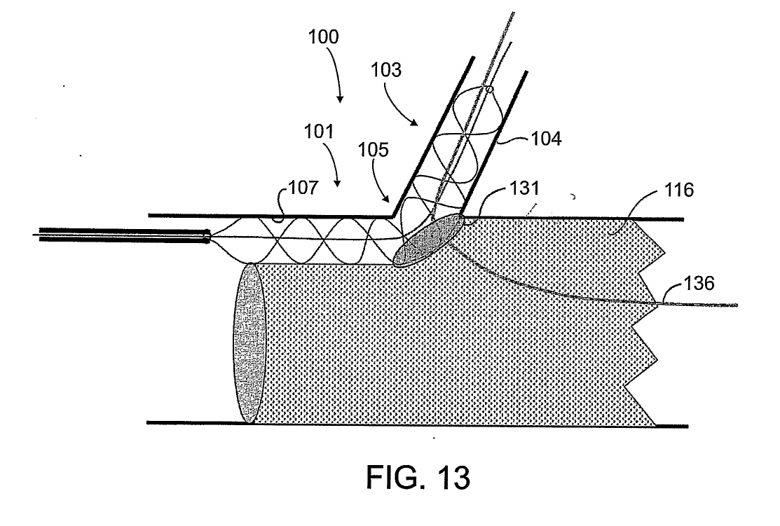

FIG. 13 shows guide wire 132 transferred from a prior position in blood vessel 102 and vessel section 101 passing through opening 131 into lumen 118, to an optionally new position where the wire is in branch vessel 104 and branch section 103 and passes through opening 131 into lumen 118. Branching scaffold 100 remains in place between stent graft 116 and inner wall 107.

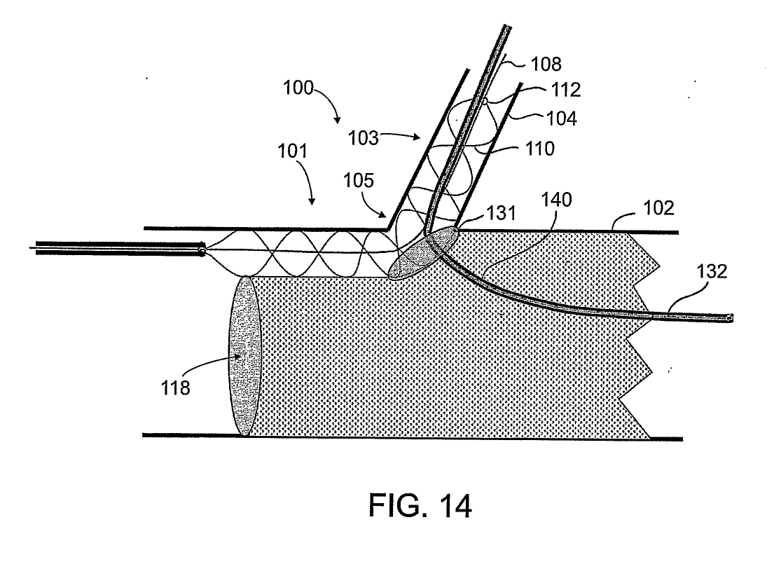

FIG. 14 shows an exemplary catheter 140 inserted through blood vessel 102 through lumen 118 and opening 131, guided by guide wire 132 into branch vessel 104. Optionally, catheter 140 is adapted to secure alignment of opening 131 with branch vessel 104. Optionally, sheath 106 operates on an exemplary slip knot 112 connecting wires 110 to the distal end of rod 108 in branching scaffold 100. Optionally, rod 108 is pulled in a proximal direction so that slips knot 112 slides out the distal end of the rod, releasing wires 110. Optionally, the structural rigidity of branching scaffold 100 is lost when wires 110 are released.

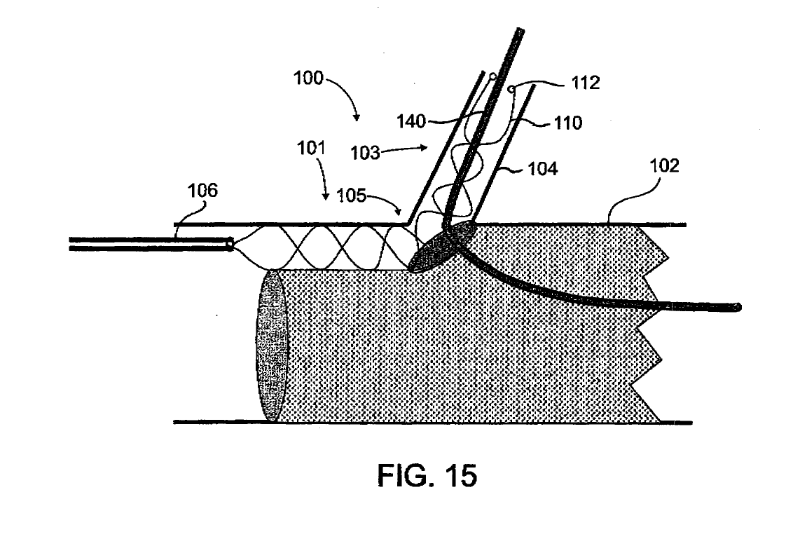

FIG. 15 shows rod 108 retrieved through sheath 106. Optionally, slip knot 112 is released (as described above) and wires 110 are disconnected. Optionally, branching scaffold 100 loses structural rigidity.

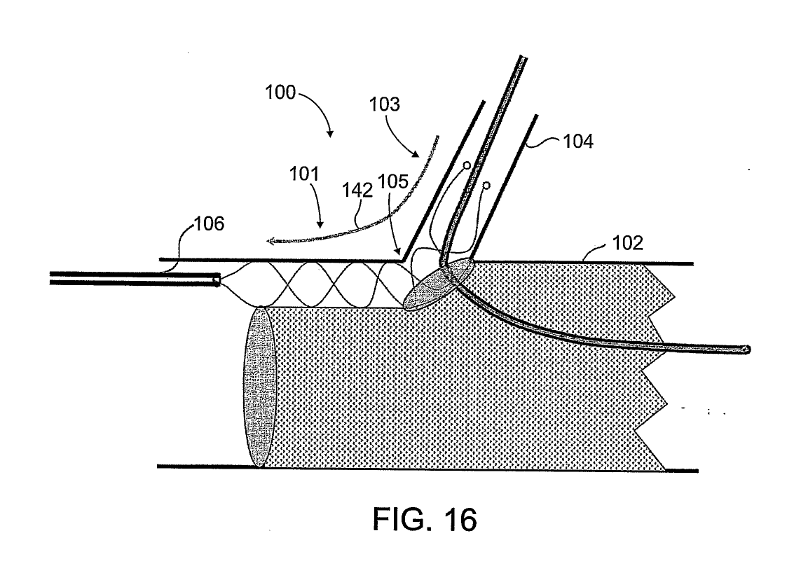

FIG. 16 shows branching scaffold 100 pulled in a proximal direction 142 out of branch vessel 104 and into blood vessel 102. Optionally, vessel section 101 and bendable section 105 are retrieved into sheath 106. Optionally, branching scaffold 100 has lost structural rigidity.

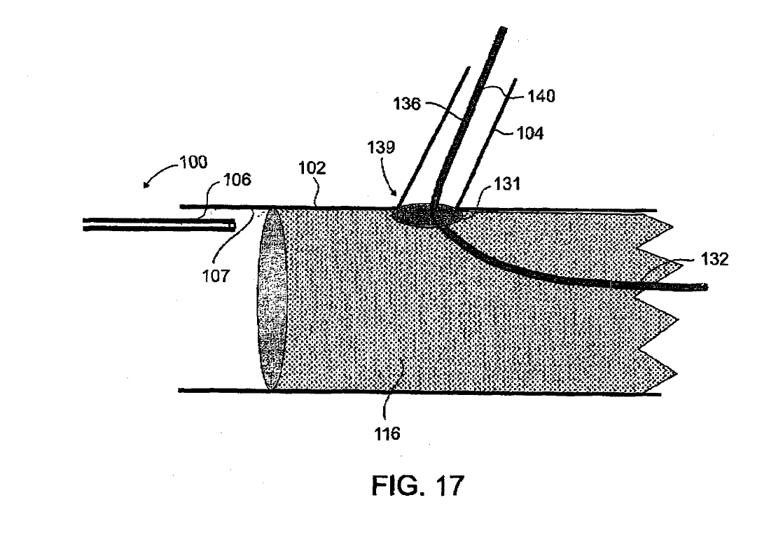

FIG. 17 shows stent graft 116 fully deployed. Optionally branching scaffold 100 is fully retrieved into sheath 106. Optionally stent graft 116 is balloon-expanded and fills a space occupied by conduit lumen 114 (see FIG. 5) abutting with inner wall 107. Optionally, opening 131 is displaced to junction 139 interconnecting branch vessels 104 and blood vessel 102. Optionally, suitable blood flow exists between blood vessel 102 and branch vessel 104 through opening 131. Optionally, catheter 140 and guide wire 132 may be removed from the side of blood vessel 102. Optionally, catheter 140 and guide wire 132 may be removed from the side of branch vessel 104. Additionally or alternatively, sheath 106 may be removed.

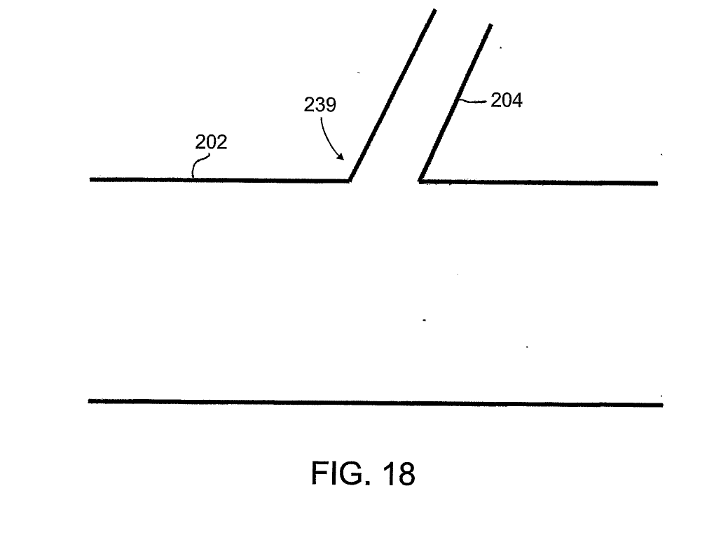

Referring now to the drawings, FIGS. 18-33 schematically illustrate an exemplary implementation of the method of deployment of an exemplary stent graft 216 in a blood vessel 202 including at least one branch vessel 204, using an exemplary stent 200, according to some embodiments of the present invention. Stent 200 may be a balloon-expandable stent. Optionally, stent 200 is a self-expanding stent. Optionally, stent 200 is expandable by expanding means other than a balloon.

FIG. 18 shows blood vessel 202 with branch vessel 204 connected to the blood vessel at junction 239. Blood vessel 202, branch vessel 204, and junction 239 may be the same as that shown in FIG. 1 at 102, 104 and 139.

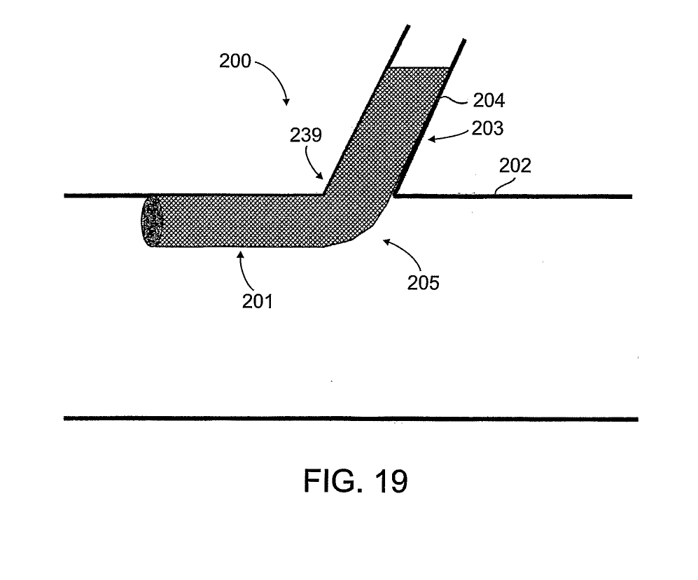

FIG. 19 shows full deployment of stent 200. Optionally, branch section 203 is deployed in branch vessel 204, an exemplary vessel section 201 is axially deployed in blood vessel 202, and an exemplary bendable section 205 joining the vessel section and branch section is deployed at junction 239. Optionally, vessel section 201 is parallel to stent graft 216.

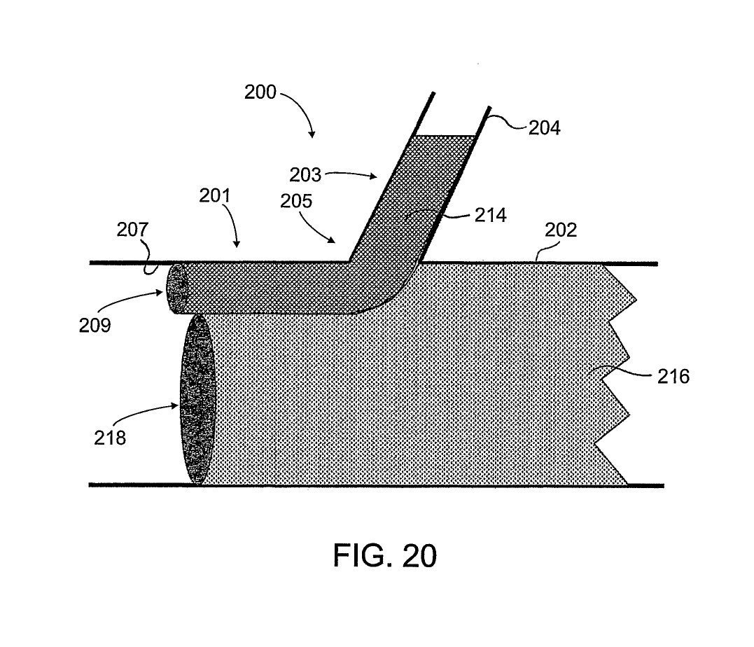

FIG. 20 shows a deployment of stent graft 216 in blood vessel 202, following deployment of stent 200. Optionally, stent graft 216 pushes vessel section 201 against an inner wall 207 of blood vessel 202. Optionally, vessel section 202 includes a structural rigidity for resisting a compressive force radially exerted on the section by stent graft 216 and inner wall 207. Stent 200 includes a conduit lumen 214 (between stent graft 216 and inner wall 207) extending from a conduit opening 209 and extending into branch vessel 204 for conducting blood flow from blood vessel 202 through junction 239 into the branch vessel. Optionally, blood flows into a lumen 218 in stent graft 216. Optionally, conduit opening 209 and an opening to stent lumen 218 in stent graft 216 are aligned.

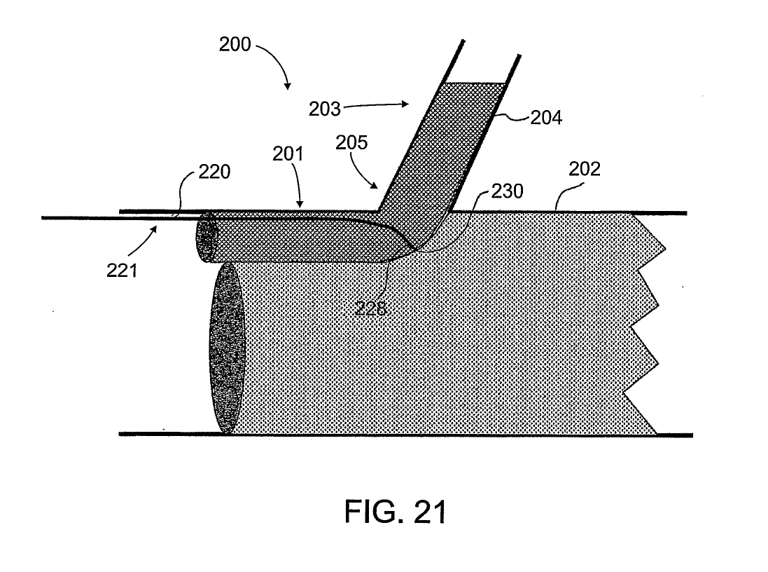

FIG. 21 shows a deployment of an exemplary fenestration device 221 for perforating a hole to form a fenestration in bendable section 205 and stent graft 216. Optionally, fenestration device 221 includes a catheter 220 which is manipulated through vessel section 201 to bendable section 205 by a guide wire 232 (see FIG. 21). Optionally, fenestration device 221 including a catheter tip 228 is guided to a fenestration location 230 in bendable section 205 where an opening is to be made. Optionally, fenestration device 221 can reach bendable section 205 from other directions, for example, from branch vessel 204. Optionally, fenestration location 230 where the opening is to be made in stent graft 216 can be reached through blood vessel 202.

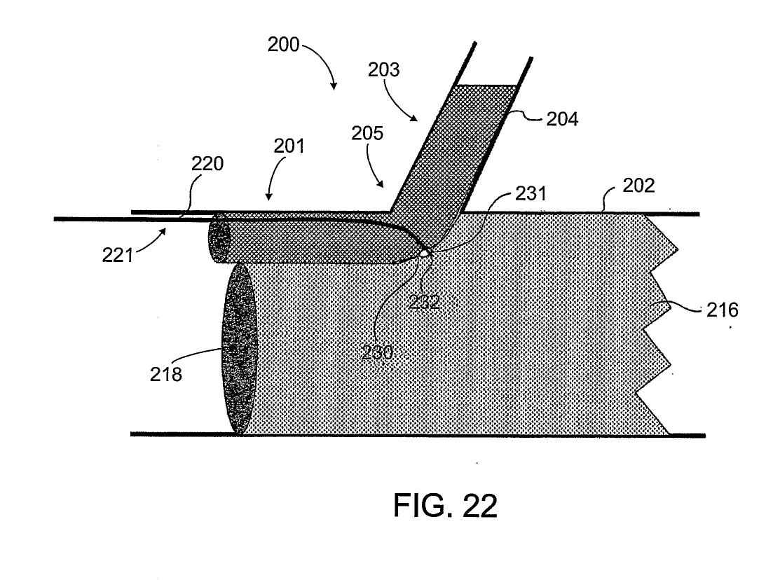

FIG. 22 shows fenestration device 221 having perforated bendable section 205 and stent graft 216 at fenestration location 230 opening an exemplary hole 231. Optionally, guide wire 232 is passed through opening 231 from catheter 220 inside vessel section 201 to lumen 218 in stent graft 216.

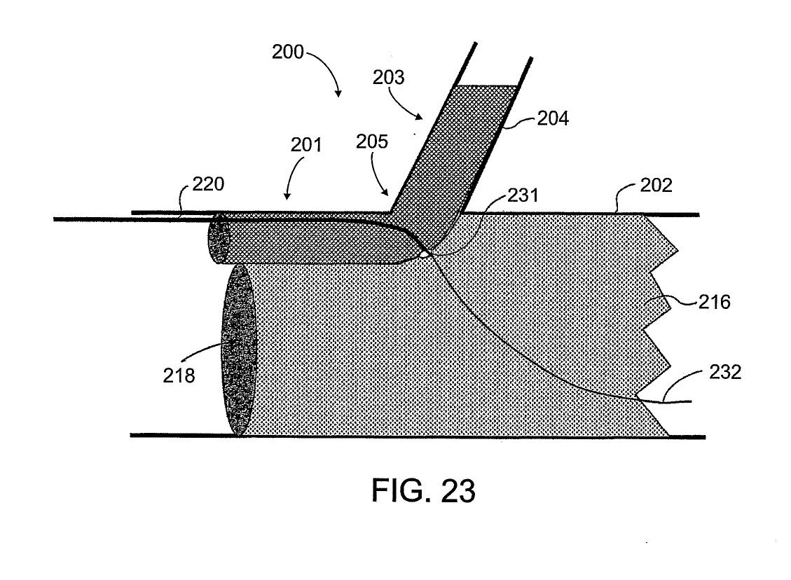

FIG. 23 shows guide wire 232 extending from inside catheter 220 through vessel section 201 and through opening 231 in bendable section 205 and stent graft 217, passing into lumen 218 in stent graft 216.

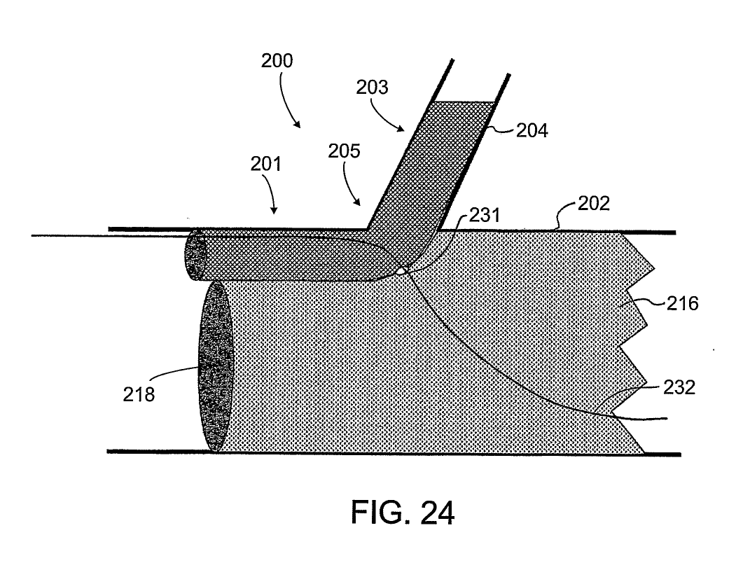

FIG. 24 shows guide wire 232 extending 220 through vessel section 201 and through opening 231 in bendable section 205 and stent graft 217, passing into lumen 218 in stent graft 216. Catheter 220 has been removed.

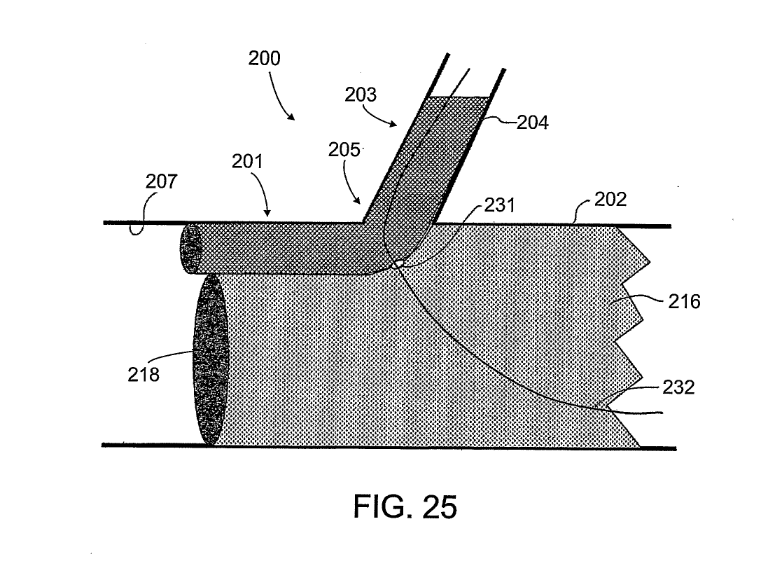

FIG. 25 shows guide wire 232 transferred from a prior position in blood vessel 202 and vessel section 201 passing through opening 231 into lumen 218, to an optionally new position where the wire is in branch vessel 204 and branch section 203 and passes through opening 231 into lumen 218. Stent 200 remains in place between stent graft 216 and inner wall 207.

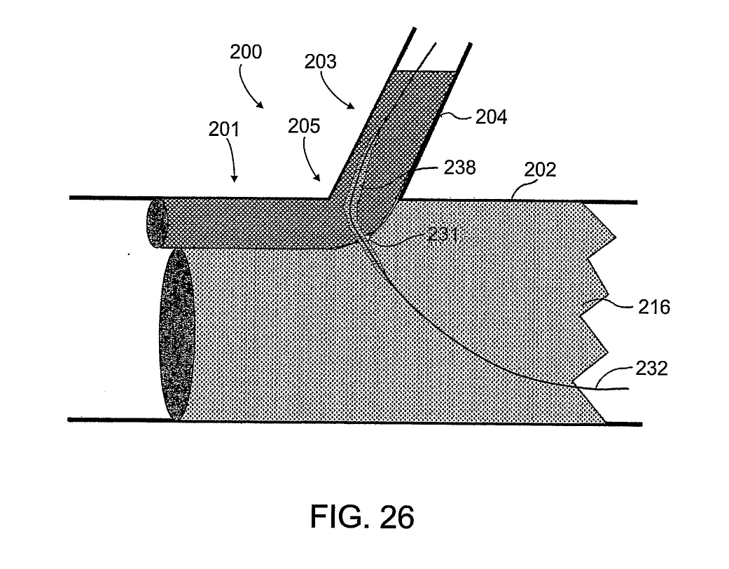

FIG. 26 shows an exemplary balloon 238 for expanding a size of opening 231 guided on guide wire 232 to the opening. Optionally, balloon 238 is inserted through blood vessel 202. Optionally, balloon 238 is inserted through branch vessel 204.

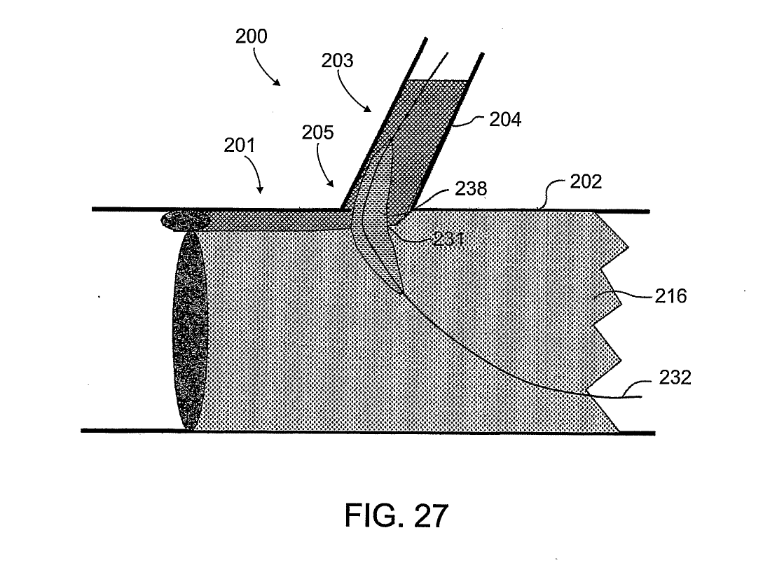

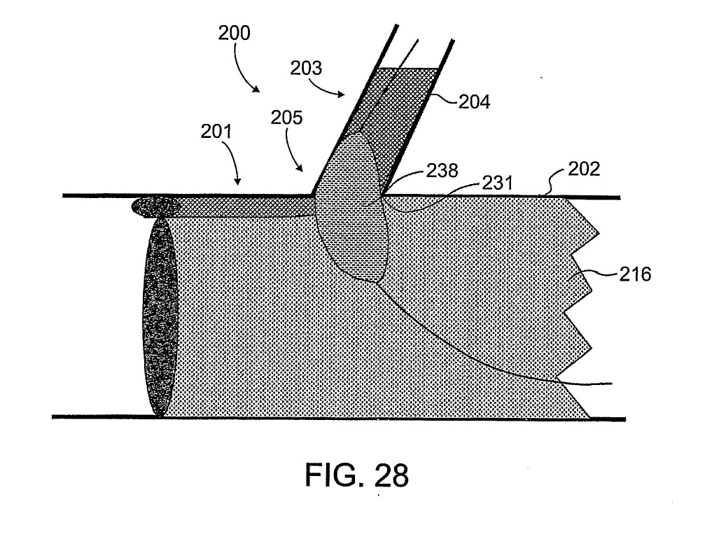

FIGS. 27 and 28 show balloon 238 being expanded inside opening 231. Optionally, balloon 238 expands opening 231 to a suitable size for allowing blood flow through the opening between branch vessel 204 and blood vessel 202. Optionally, blood flow through vessel section 201 and bendable section 205 into branch section 203 is interrupted.

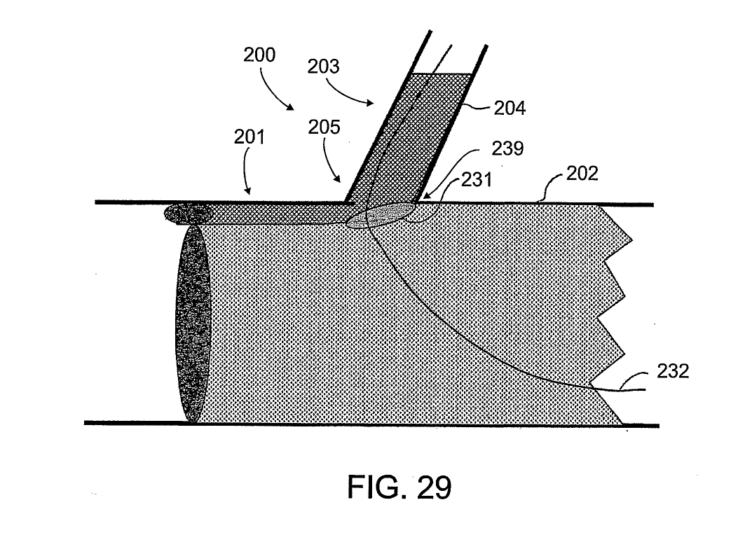

FIG. 29 shows an exemplary suitable opening 231 for allowing blood flow between branch vessel 204 and blood vessel 202. Optionally, balloon 238 is deflated and retrieved along guide wire 232. Optionally, balloon 238 is retrieved through blood vessel 202. Additionally or alternatively, balloon 238 is retrieved through branch vessel 204. Optionally, guide wire 232 remains in place, passing through branch section 203 and through opening 231 and into lumen 218 in stent graft 216.

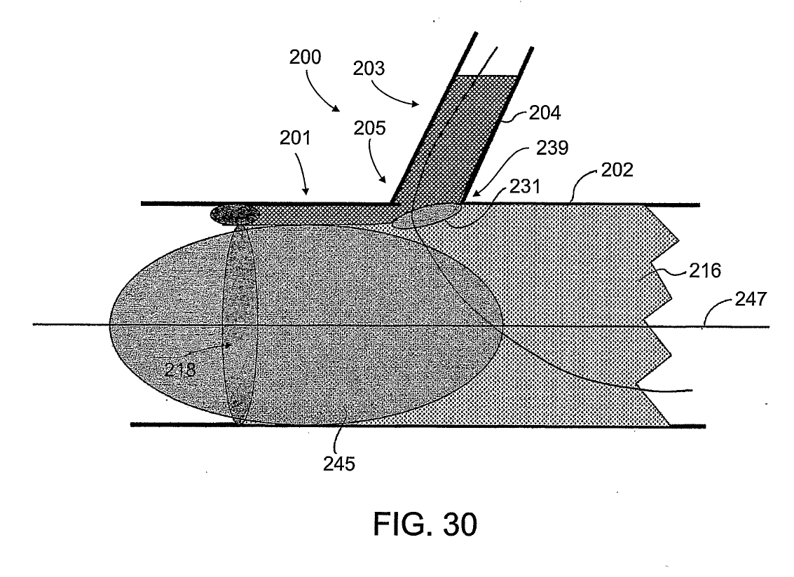

FIG. 30 shows an exemplary balloon 245 for expanding stent graft 216 inserted in blood vessel 202. Optionally, a guide wire 247 is in guided through blood vessel 202 into lumen 218. Optionally, balloon 245 is guided along guide wire 247 to a correct position for expanding stent graft 216. Optionally, balloon 245 is positioned so that, when expanded, vessel section 201 collapses under the compressive force of stent graft 216 on the vessel section.

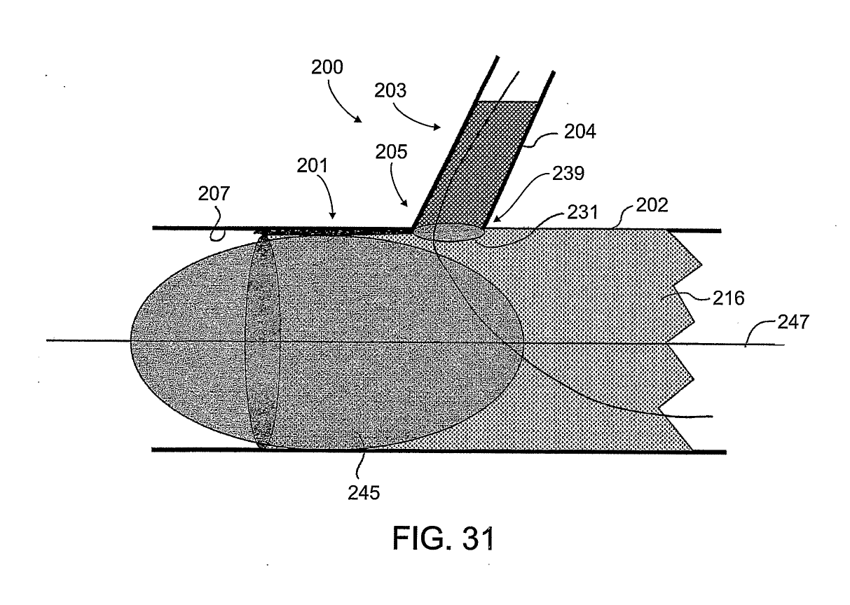

FIG. 31 shows balloon 245 expanded inside stent graft 216, causing vessel section 201 to collapse under the compressive force of the balloon-expanded stent. Optionally, vessel section 201 collapses into a relatively thin layer between inner wall 206 and balloon-expanded stent graft 216. Optionally, balloon 245 may be deflated following expansion of stent graft 216 and removed along guide wire 247. Optionally, guide wire 247 may be removed.

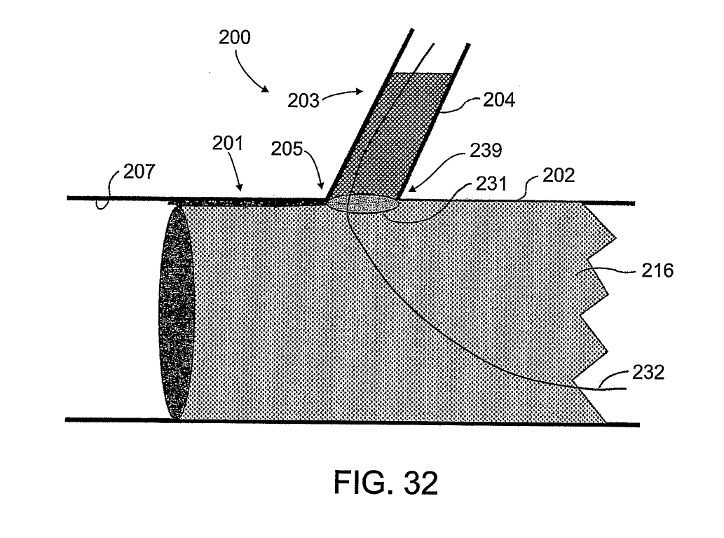

FIG. 32 shows stent graft 216 fully deployed. Optionally stent graft 216 fills a space occupied by vessel section 201 to inner wall 207 (except for the crushed vessel section 201 between the wall and the stent graft). Optionally, opening 231 is displaced to junction 239 interconnecting branch vessels 204 and blood vessel 202. Optionally, suitable blood flow exists between blood vessel 202 and branch vessel 204 through opening 231. Optionally, guide wire 232 may be removed from the side of blood vessel 102. Optionally, guide wire 132 may be removed from the side of branch vessel 204.

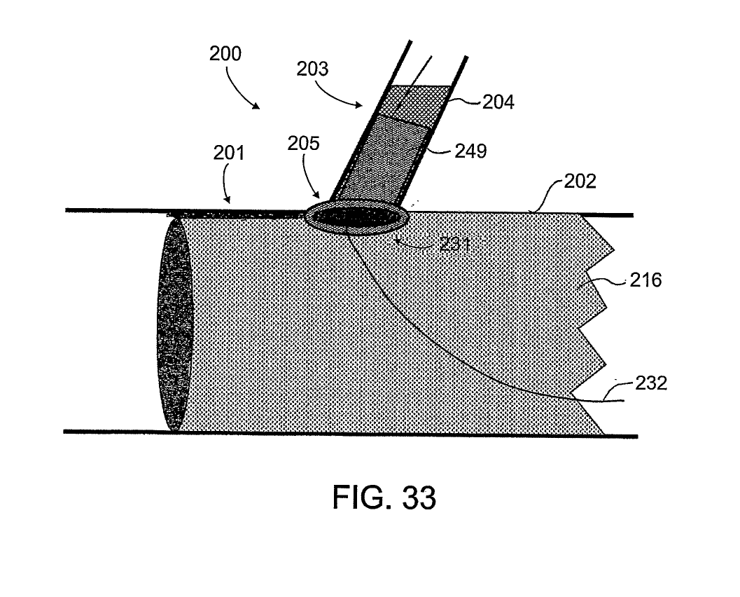

FIG. 33 shows an optional branch stent graft 249 inserted in branch vessel 204. Optionally, branch stent graft 249 is advanced over guide wire 232 through stent graft 216, through fenestration 231 and into branch section 204. Optionally, branch stent graft 249 is expanded by unsheathing, balloon expansion or other stent expansion methods. Optionally, branch stent graft 249 is mounted over fenestration 231. Optionally, branch stent graft 249 is inserted from a direction of branch vessel 204.

The foregoing descriptions of exemplary implementations of the method are not intended to be limiting in any way, form or manner. It should be evident to an ordinary person skilled in the art that there are numerous other ways, form, or manner of implementing the method. These may include adding steps, deleting steps, skipping steps, and changing a sequence of steps. Furthermore, it should be evident to a person skilled in art that foregoing descriptions of insertion and removal of catheters and/or guide wires, and of expanding stents and stent grafts, are for exemplary purposes only, and that these may be varied, for example, according to established medical practice.

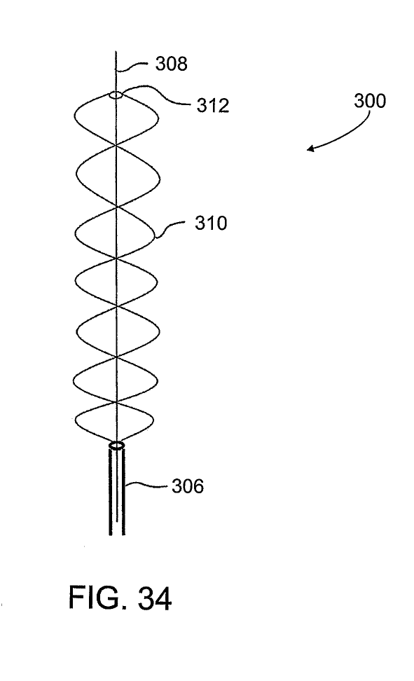

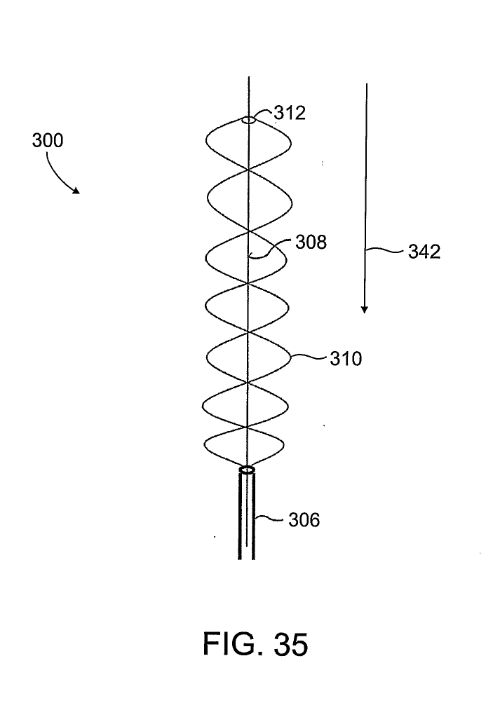

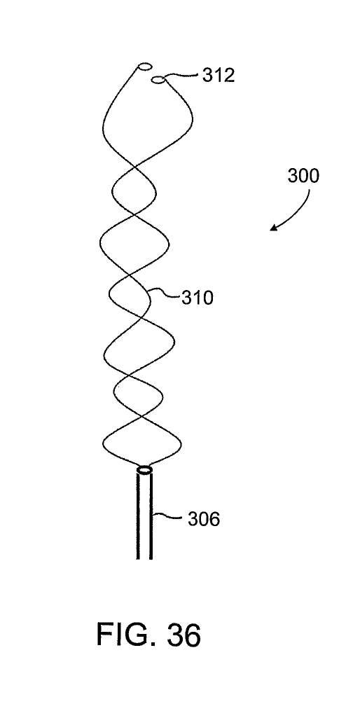

Reference is now made to FIGS. 34-36 which schematically illustrate an exemplary branching scaffold 300 and a method of loss of structural rigidity in the scaffold, according to some embodiments of the present invention. Branching scaffold 300 includes a sheath 306, a rod 308, and wires 310 adapted to serve as struts and connected to the rod in a loop 312 (slip knot) at a distal end of the rod. Branching scaffold 300 including sheath 306, rod 308, wires 310, and loop 312 may be similar to that shown in FIGS. 1-17 at 100, 106, 108, 110, and 112. Branching scaffold 300 loses structural rigidity when rod 308 is pulled in a proximal direction 342, loop 312 sliding out of the rod releasing wires 310. Optionally, rod 308 and wires 310 can be retrieved into sheath 306.

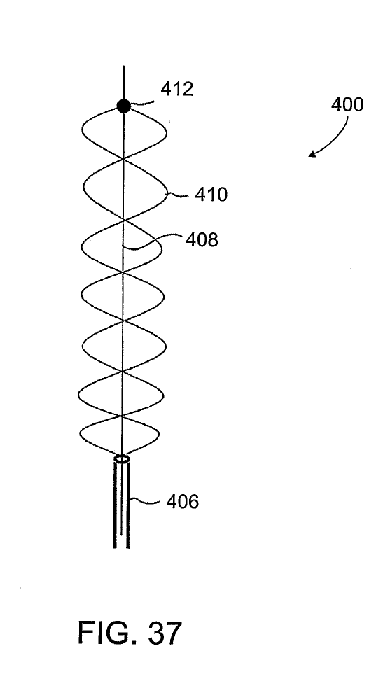

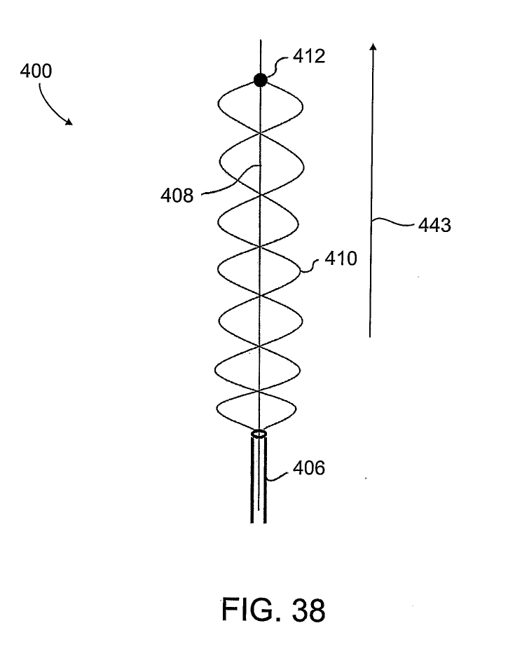

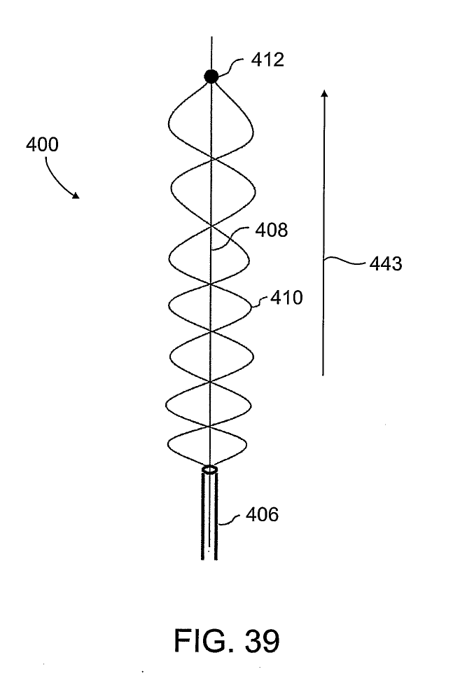

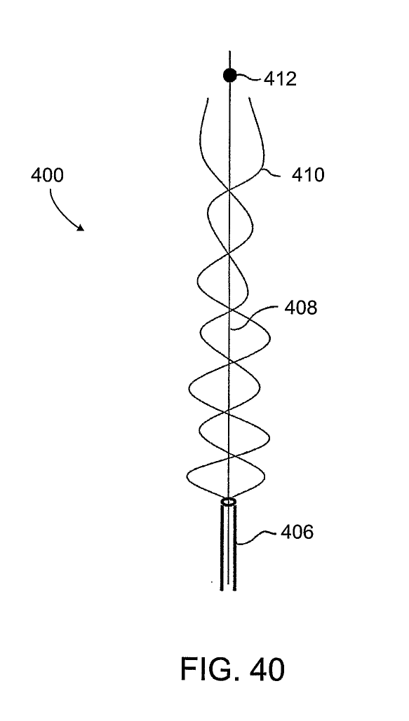

Reference is now made to FIGS. 37-40 which schematically illustrate an exemplary branching scaffold 400 and a method of loss of structural rigidity in the scaffold, according to some embodiments of the present invention. Branching scaffold 400 includes a sheath 406, a rod 408, and wires 410 adapted to serve as struts and connected to the rod in a joint 412 at a distal end of the rod. Branching scaffold 400 including sheath 406, rod 408, and wires 410 may be similar to that shown in FIGS. 1-17 at 100, 106, 108, and 110 with an optional difference that branching scaffold 400 includes joint 412. Branching scaffold 400 loses structural rigidity when rod 408 is pushed in a distal direction 443, the rod pushing joint 412 in the distal direction causing wires 410 to break loose from the joint. Optionally, rod 408 and wires 410 can be retrieved into sheath 406.

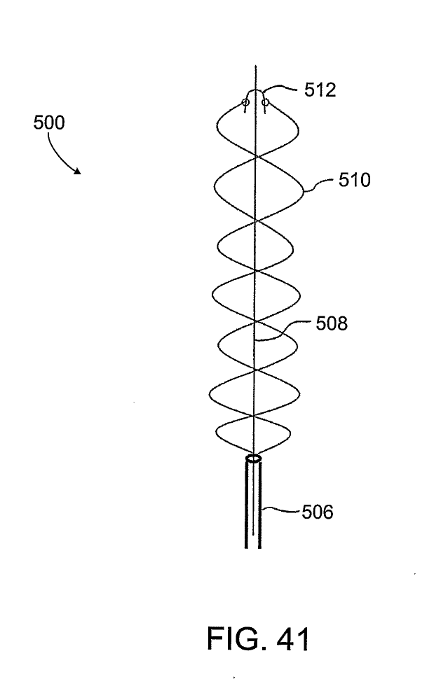

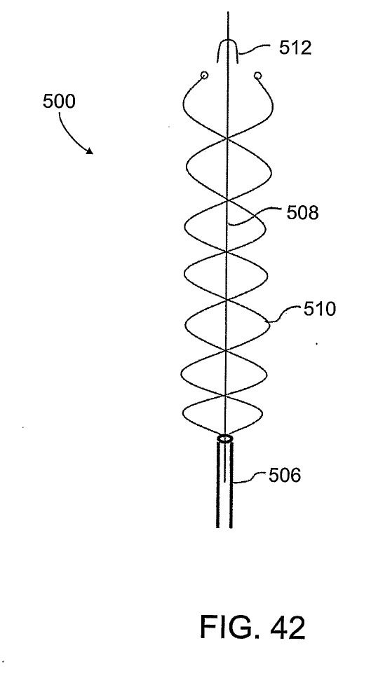

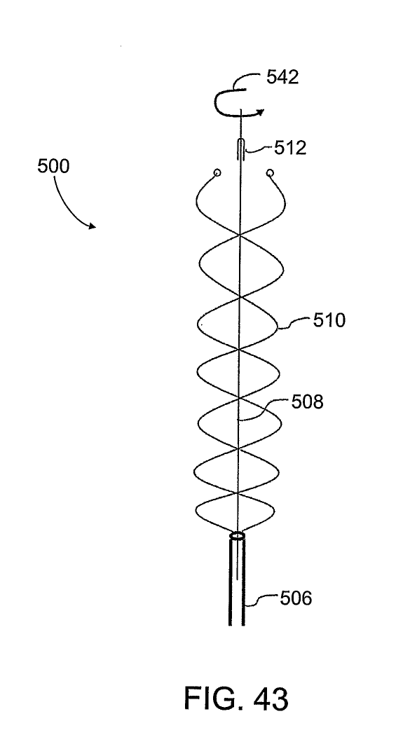

Reference is now made to FIGS. 41-43 which schematically illustrate an exemplary branching scaffold 500 and a method of loss of structural rigidity in the scaffold, according to some embodiments of the present invention. Branching scaffold 500 includes a sheath 506, a rod 508, and wires 510 adapted to serve as struts and connected to the rod in a hook 512 at a distal end of the rod. Branching scaffold 500 including sheath 506, rod 508, and wires 510 may be similar to that shown in FIGS. 1-17 at 100, 106, 108, and 110, with an optional difference that branching scaffold 500 includes hook 512. Branching scaffold 500 loses structural rigidity when rod 508 is turned in a twisting direction 542 causing wires 510 to slide off hook 512. Optionally, rod 508 and wires 510 can be retrieved into sheath 506.

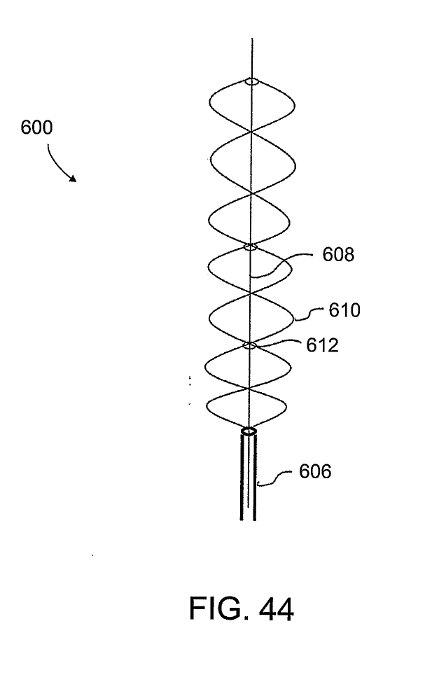

Reference is now made to FIG. 44 which schematically illustrates an exemplary branching scaffold 600, according to some embodiments of the present invention. Branching scaffold 600 includes a sheath 606, a rod 608, and wires 610 adapted to serve as struts and connected to the rod in a plurality of loops 612 (slip knot). Branching scaffold 600 including sheath 606, rod 608, wires 610, and loops 612 may be similar to that shown in FIGS. 1-17 at 100, 106, 108, 110, and 112, with an optional difference that branching scaffold 600 includes a plurality of loops. Optionally, rod 608 and wires 610 can be retrieved into sheath 606.

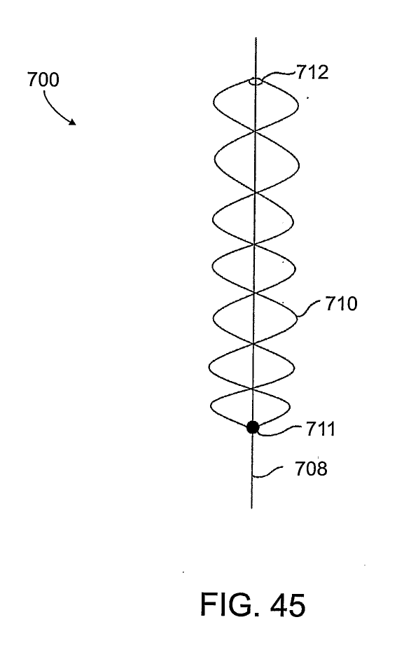

Reference is now made to FIG. 45 which schematically illustrates an exemplary branching scaffold 700, according to some embodiments of the present invention. Branching scaffold 700 includes a sheath 706, a rod 708, wires 710 adapted to serve as struts and connected to the rod in a loop 712 (slip knot) at a distal end of the rod, and a connection point 711 for the wires at a proximal end of the wires. Branching scaffold 700 including sheath 706, rod 708, and wires 710, and loops 712 may be similar to that shown in FIGS. 1-17 at 100, 106, 108, 110, and 112, with an optional difference that branching scaffold 700 includes connection point 711. Optionally, rod 708 and wires 710 can be retrieved into sheath 706.

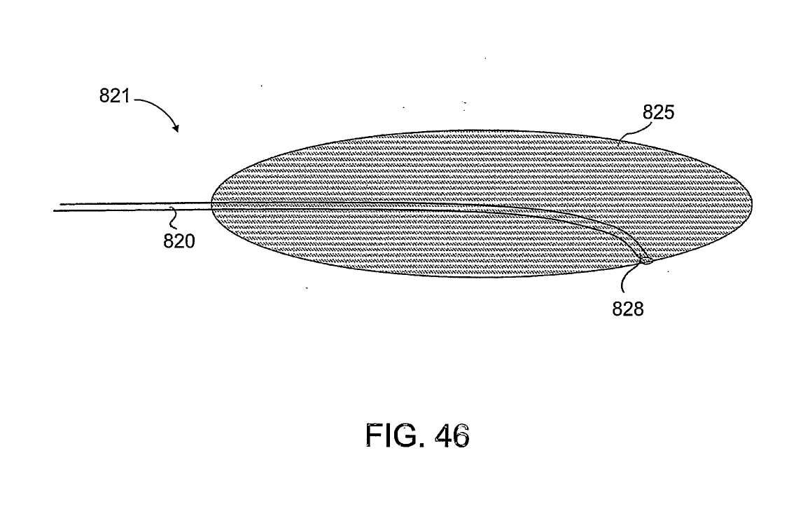

Reference is now made to FIG. 46 which schematically illustrates a fenestration device 821, according to some embodiments of the present invention. Fenestration device 821, includes a catheter 820 and a balloon 825 adapted to bend a catheter tip 828 when inflated. Fenestration device 821 including catheter 820 with catheter tip 828 and balloon 825 may be similar to that shown in FIGS. 1-17 at 121, 120, 128 and 125 with an optional difference that balloon 825 is a single balloon.

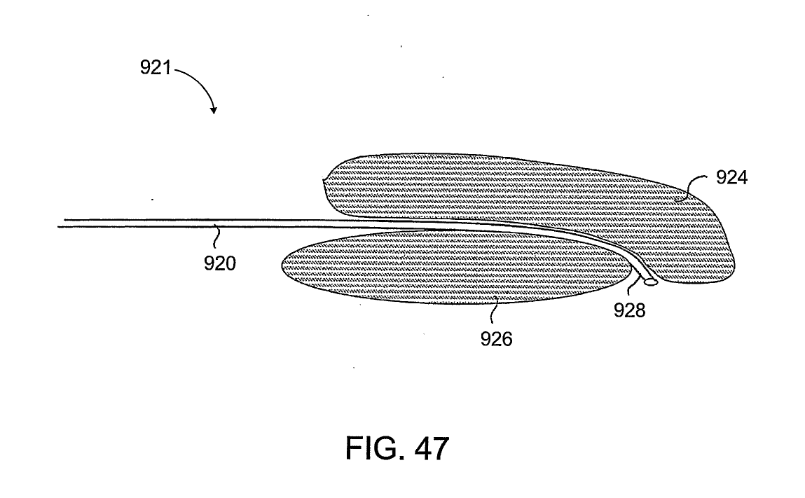

Reference is now made to FIG. 47 which schematically illustrates a fenestration device 921, according to some embodiments of the present invention. Fenestration device 921 includes a catheter 920, and a small balloon 926 and a large balloon 924 adapted to bend a catheter tip 928 when inflated. Fenestration device 921 including catheter 920 with catheter tip 928 and balloons 824 and 826 may be similar to that shown in FIGS. 1-17 at 121, 120, 128, 124 and 126.

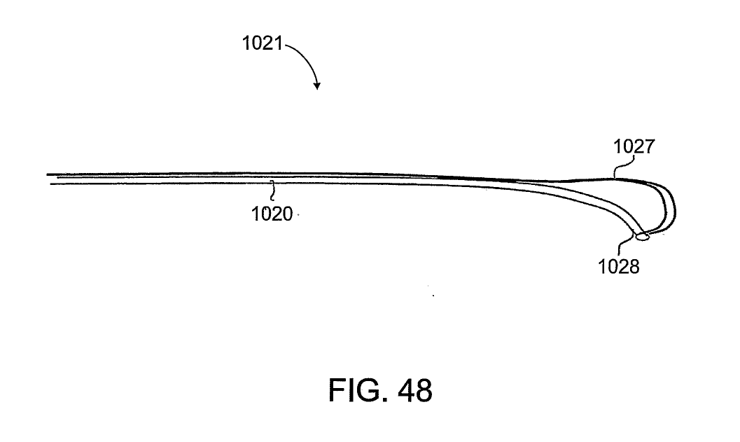

Reference is now made to FIG. 48 which schematically illustrates a fenestration device 1021, according to some embodiments of the present invention. Fenestration device 1021, includes a catheter 1020 with one or more wires 1027 running along one aspect of its wall and extending to a catheter tip 1028. Optionally, catheter tip 1028 is bent to a side when wires 1027 are pushed forward in a distal direction. Optionally, catheter tip 1028 is bent when wire 1027 is pulled in a proximal direction. Fenestration device 1021 including catheter 1020 with catheter tip 1028 may be similar to that shown in FIGS. 1-17 at 121, 120, and 128 with an optional difference that branching scaffold 1000 includes wire 1027 for bending the catheter tip.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, an and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of deploying a stent graft in a blood vessel with at least one side branch vessel, comprising:
(a) forming a blood flow conduit between the branch vessel and the blood vessel such that a vessel section of the conduit is axially located inside the blood vessel and a branch section is positioned inside the branch vessel;
(b) deploying the stent graft inside the blood vessel so that blood flow into said at least one side branch is blocked by the stent graft and continues through the blood flow conduit;
(c) forming an opening suitable for allowing blood flow between the blood vessel and the branch vessel; and
(d) interrupting blood flow in the vessel section of the conduit.

2. The method of claim 1 comprising forming the blood flow conduit with a stent adapted to transport blood from said blood vessel to said at least one side branch vessel.

3-4. (canceled)

5. The method of claim 1 comprising parallely locating the vessel section between an inner wall of the blood vessel and the deployed stent graft.

6. (canceled)

7. The method of claim 1 comprising interrupting blood flow in the vessel section by collapsing the vessel section.

8. The method of claim 7 comprising collapsing the vessel section under a compressive force exerted by the deployed stent graft when the stent graft is expanded.

9-15. (canceled)

16. The method of claim 1 comprising forming the opening by initially inserting a fenestration device through the vessel section and making a perforation in said conduit and said stent graft.

17. (canceled)

18. The method of claim 16 further comprising inserting an expansion balloon into the perforation and expanding the balloon to increase the size of the perforation to that of the opening.

19-22. (canceled)

23. A blood flow conductor comprising a vessel section and a branch section connected together by a bendable section, the blood flow conductor configured to provide blood flow to a branch vessel bypassing a deployed stent graft, wherein at least the vessel section is adapted to be deployed in the aorta and maintains a structural rigidity when compressed between the deployed stent graft and the aortic wall for maintaining a blood flow through the blood flow conductor, and wherein said vessel section is collapsible for interrupting said blood flow.

24. The blood flow conductor of claim 23 comprising a stent.

25. The blood flow conductor of claim 24 wherein said stent is a self-expanding stent.

26. (canceled)

27. (canceled)

28. (canceled)

29. The blood flow conductor of claim 25 comprising a sheath for transporting said stent to a location for deployment in the aorta.

30. The blood flow conductor of claim 29 wherein said stent is adapted to be folded into the sheath following collapse of at least the vessel section.

31. The blood flow conductor of claim 24 wherein the stent is a balloon-expandable stent.

32. The blood flow conductor of claim 31 wherein the vessel section of the stent is pliable.

33. (canceled)

34. The blood flow conductor of claim 23 wherein the branch section is adapted to be inserted in a branch vessel.

35. (canceled)

36. The blood flow conductor of claim 23 wherein the bendable section is kink-resistant.

37. The blood flow conductor of claim 23 comprising a wire adapted to collapse the vessel section when pulled in a proximal direction.

38. The blood flow conductor of claim 23 comprising a sliding knot for collapsing the vessel section.

39. The blood flow conductor of claim 23 comprising a breakable joint for collapsing the vessel section.

40. The blood flow conductor of claim 23 comprising a hook for collapsing the vessel section.

41-42. (canceled)

43. A medical kit comprising:
(a) an aortic stent graft;
(b) a blood flow conductor comprising a vessel section and a branch section connected together by a bendable section, the blood flow conductor configured to provide blood flow to a branch vessel bypassing a deployed stent graft, wherein at least the vessel section is adapted to be deployed in the aorta and maintains a structural rigidity when compressed between the deployed stent graft and the aortic wall, for maintaining a blood flow through the blood flow conductor, and wherein said vessel section is collapsible for interrupting said blood flow.
(c) a fenestration device for making said fenestration in said stent graft and said bendable section.

44-52. (canceled)

53. The blood flow conductor of claim 23 wherein said branch section is collapsible.

54. The blood flow conductor of claim 23 wherein the blood flow conductor is adapted to be removed from the branch vessel.

55. The blood flow conductor of claim 23 wherein the stent graft includes a substantially flow impervious wall.

56. The blood flow conductor of claim 23 wherein said bendable section is crimp-resistant for substantially maintaining blood flow uninterrupted through the blood flow conductor.
